(19) United States
(12) Patent Application Publication
(10) Pub. No.: US 2014/0343128 A1
(43) Pub. Date: Nov. 20, 2014

(54) Combination of a phosphoinositide 3-kinase inhibitor and a modulator of the Janus Kinase 2 - Signal Transducer and Activator of Transcription 5 pathway

(71) Applicant: NOVARTIS FORSCHUNGSSTIFTUNG, ZWEIGNIEDERLASSUNG FRIEDRICH MIESCHER INSTITUTE FOR BIOMEDICAL RESEAR, Basel (CH)

(72) Inventors: Mohamed Bentires-Alj, Kembs-Loechle (FR); Adrian Britschgi, Hergiswil (CH)

(73) Assignee: Novartis AG, Basel (CH)

(21) Appl. No.: 14/357,596

(22) PCT Filed: Nov. 14, 2012
(86) PCT No.: PCT/EP2012/072657

§ 371 Date: May 13, 2014

(30) Foreign Application Priority Data
Nov. 15, 2011 (EP) 11189194.1

(51) Int. Cl. A61K 31/5377; A61K 31/436; A61K 31/4745

(52) U.S. Cl. CPC A61K 31/5377; A61K 31/4745; A61K 31/436
USPC 514/44 A; 514/234.8; 514/232.2

(57) ABSTRACT

The invention relates to a pharmaceutical combination which comprises (a) a phosphoinositide 3-kinase inhibitor compound and (b) a compound which modulates the JAK2-STAT5 pathway for the treatment of a proliferative disease, especially a solid tumor disease; a pharmaceutical composition comprising such a combination; the use of such a combination for the preparation of a medicament for the treatment of a proliferative disease; a commercial package or product comprising such a combination as a combined preparation for simultaneous, separate or sequential use; and to a method of treatment of a warm-blooded animal, especially a human.

The invention relates to a pharmaceutical combination which comprises (a) a phosphoinositide 3-kinase (PI3K) inhibitor compound and (b) a compound which modulates the Janus Kinase 2 (JAK2)-Signal Transducer and Activator of Transcription 5 (STAT5) pathway and optionally at least one pharmaceutically acceptable carrier for simultaneous, separate or sequential use, in particular for the treatment of a proliferative disease, especially a proliferative disease in which the PI3K/Akt pathway is concomitantly dysregulated; a pharmaceutical composition comprising such a combination; the use of such a combination for the preparation of a medicament for the treatment of a proliferative disease; a commercial package or product comprising such a combination as a combined preparation for simultaneous, separate or sequential use; and to a method of treatment of a warm-blooded animal, especially a human.

The rapid development of highly specific inhibitors targeting key signaling pathways (e.g., PI3K/mTOR) has created much excitement in the cancer research community. The clinical efficacy and low toxicity of some of these rationally designed therapies raised the hope for a new era for the treatment of cancer. Unfortunately, single-agent targeted cancer therapy is often thwarted by adaptive resistance, tumor recurrence and an ineluctable downhill course. A better understanding of the crosstalks between oncogenic signaling pathways is fundamental to curb resistance to targeted therapy and should lead to novel, hopefully curative, combination therapies.

The phosphatidylinositol 3-kinase (PI3K) pathway, a central regulator of diverse normal cellular functions, is often subverted during neoplastic transformation. Mechanisms of activation of the PI3K pathway in cancer include: mutation and/or amplification of PIK3CA, the gene encoding p110α, the alpha catalytic subunit of the kinase; loss of expression of PTEN, the phosphatase that reverses PI3K activity; activation downstream of oncogenic receptor tyrosine kinases; and Akt amplification. By decreasing cell death, increasing cell proliferation, migration, invasion, metabolism, angiogenesis and resistance to chemotherapy, an aberrant PI3K pathway provides cancer cells with a competitive advantage. Not surprisingly, the PI3K/Akt/mTOR cascade is an attractive therapeutic target and several inhibitors of this pathway are currently in clinical trials.

Using several cell lines and primary tumor models of triple-negative breast cancer, the present inventors found that PI3K/mTOR inhibition elicited a vicious positive feedback loop by activating JAK2-STAT5 signaling which induced secretion of IL-8, a chemotactic cytokine with crucial roles in metastasis. IL-8 in turn fed back into JAK2/STAT5, thereby completing the loop. Notably, inducible JAK2 shRNAs and a JAK2 inhibitor abrogated this feedback and reduced tumor seeding and metastasis.

Building on insights gained from mechanistic understanding of PI3K/mTOR inhibition, the present inventors demonstrated the therapeutic efficacy of combined inhibition of the PI3K/mTOR and JAK2-STAT5 pathways. Indeed combined inhibition of PI3K/mTOR and JAK2-STAT5 reduced tumor growth and seeding as well as metastasis.

WO2006/122806 describes imidazoquinoline derivatives, which have been described to inhibit the activity of lipid kinases, such as PI3-kinases. Specific imidazoquinoline derivatives which are suitable for the present invention, their preparation and suitable pharmaceutical formulations containing the same are described in WO2006/122806 and include compounds of formula I

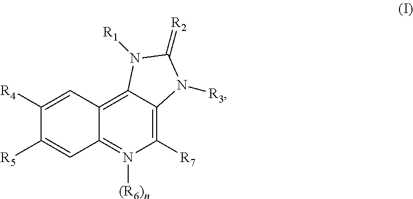

wherein

R₁ is naphthyl or phenyl wherein said phenyl is substituted by one or two substituents independently selected from the group consisting of Halogen; lower alkyl unsubstituted or substituted by halogen, cyano, imidazolyl or triazolyl; cycloalkyl; amino substituted by one or two substituents independently selected from the group consisting of lower alkyl, lower alkyl sulfonyl, lower alkoxy and lower alkoxy lower alkylamino; piperazinyl unsubstituted or substituted by one or two substituents independently selected from the group consisting of lower alkyl and lower alkyl sulfonyl; 2-oxo-pyrrolidinyl; lower alkoxy lower alkyl; imidazolyl;

pyrazolyl; and triazolyl;

R₂ is O or S;

R₃ is lower alkyl;

R₄ is pyridyl unsubstituted or substituted by halogen, cyano, lower alkyl, lower alkoxy or piperazinyl unsubstituted or substituted by lower alkyl; pyrimidinyl unsubstituted or substituted by lower alkoxy; quinolinyl unsubstituted or substituted by halogen;

quinoxalinyl; or phenyl substituted with alkoxy

R₅ is hydrogen or halogen;

n is 0 or 1;

R₆ is oxido;

with the proviso that if n=1, the N-atom bearing the radical R₆ has a positive charge;

R₇ is hydrogen or amino;

or a tautomer thereof, or a pharmaceutically acceptable salt, or a hydrate or solvate thereof.

The radicals and symbols as used in the definition of a compound of formula I have the meanings as disclosed in WO2006/122806 which publication is hereby incorporated into the present application by reference.

A compound of the present invention is a compound which is specifically described in WO2006/122806. A compound of the present invention is 2-methyl-2-[4-(3-methyl-2-oxo-8-quinolin-3-yl-2,3-dihydro-imidazo[4,5-c]quinolin-1-yl)-phenyl]-propionitrile and its monotosylate salt (COMPOUND A, also known as BEZ-235). The synthesis of 2-methyl-2-[4-(3-methyl-2-oxo-8-quinolin-3-yl-2,3-dihydro-imidazo[4,5-c]quinolin-1-yl)-phenyl]-propionitrile is for instance described in WO2006/122806 as Example 7. Another compound of the present invention is 8-(6-methoxy-pyridin-3-yl)-3-methyl-1-(4-piperazin-1-yl-3-trifluoromethyl-phenyl)-1,3-dihydro-imidazo[4,5-c]quinolin-2-one (COMPOUND B). The synthesis of 8-(6-methoxy-pyridin-3-yl)-3-methyl-1-(4-piperazin-1-yl-3-trifluoromethyl-phenyl)-1,3-dihydro-imidazo[4,5-c]quinolin-2-one is for instance described in WO2006/122806 as Example 86.

WO07/084786 describes pyrimidine derivatives, which have been found to inhibit the activity of lipid kinases, such as PI3-kinases. Specific pyrimidine derivatives which are suitable for the present invention, their preparation and suitable pharmaceutical formulations containing the same are described in WO07/084786 and include compounds of formula II

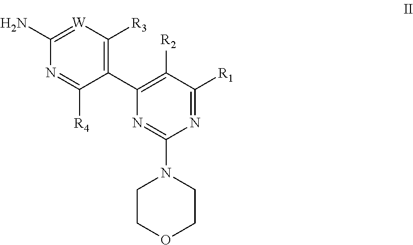

or a stereoisomer, tautomer, or pharmaceutically acceptable salt thereof, wherein,
W is CR_{w} or N, wherein R_{w} is selected from the group consisting of
(1) hydrogen,
(2) cyano,
(3) halogen,
(4) methyl,
(5) trifluoromethyl,
(6) sulfonamido;
R₁ is selected from the group consisting of
(1) hydrogen,
(2) cyano,
(3) nitro,
(4) halogen,
(5) substituted and unsubstituted alkyl,
(6) substituted and unsubstituted alkenyl,
(7) substituted and unsubstituted alkynyl,
(8) substituted and unsubstituted aryl,
(9) substituted and unsubstituted heteroaryl,
(10) substituted and unsubstituted heterocyclyl,
(11) substituted and unsubstituted cycloalkyl,
(12) —COR₁ₐ,
(13) —CO₂R₁ₐ,
(14) —CONR₁ₐR_{1b},
(15) —NR₁ₐR_{1b},
(16) —NR₁ₐCOR_{1b},
(17) —NR₁ₐSO₂R_{1b},
(18) —OCOR₁ₐ,
(19) —OR₁ₐ,
(20) —SR₁ₐ,
(21) —SOR₁ₐ,
(22) —SO₂R₁ₐ, and
(23) —SO₂NR₁ₐR_{1b},
wherein R₁ₐ, and R_{1b} are independently selected from the group consisting of
(a) hydrogen,
(b) substituted or unsubstituted alkyl,
(c) substituted and unsubstituted aryl,
(d) substituted and unsubstituted heteroaryl,
(e) substituted and unsubstituted heterocyclyl, and
(f) substituted and unsubstituted cycloalkyl;
R₂ is selected from the group consisting
(1) hydrogen,
(2) cyano,
(3) nitro,
(4) halogen,
(5) hydroxy,
(6) amino,
(7) substituted and unsubstituted alkyl,
(8) —COR₂ₐ, and
(9) —NR₂ₐCOR_{2b},
wherein R₂ₐ, and R_{2b} are independently selected from the group consisting of
(a) hydrogen, and
(b) substituted or unsubstituted alkyl;
R₃ is selected from the group consisting of
(1) hydrogen,
(2) cyano,
(3) nitro,
(4) halogen,
(5) substituted and unsubstituted alkyl,
(6) substituted and unsubstituted alkenyl,
(7) substituted and unsubstituted alkynyl,
(8) substituted and unsubstituted aryl,
(9) substituted and unsubstituted heteroaryl,
(10) substituted and unsubstituted heterocyclyl,
(11) substituted and unsubstituted cycloalkyl,
(12) —COR₃ₐ,
(13) —NR₃ₐR_{3b},
(14) —NR₃ₐCOR_{3b},
(15) —NR₃ₐSO₂R_{3b},
(16) —OR₃ₐ,
(17) —SR₃ₐ,
(18) —SOR₃ₐ,
(19) —SO₂R₃ₐ, and
(20) —SO₂NR₃ₐR_{3b},
wherein R₃ₐ, and R_{3b} are independently selected from the group consisting of
(a) hydrogen,
(b) substituted or unsubstituted alkyl,
(c) substituted and unsubstituted aryl,
(d) substituted and unsubstituted heteroaryl,
(e) substituted and unsubstituted heterocyclyl, and
(f) substituted and unsubstituted cycloalkyl; and
R₄ is selected from the group consisting of
(1) hydrogen, and
(2) halogen.

The radicals and symbols as used in the definition of a compound of formula II have the meanings as disclosed in WO07/084786 which publication is hereby incorporated into the present application by reference.

A compound of the present invention is a compound which is specifically described in WO07/084786.

A compound of the present invention is 5-(2,6-di-morpholin-4-yl-pyrimidin-4-yl)-4-trifluoromethyl-pyridin-2-ylamine (COMPOUND C, also known as BKM-120). The synthesis of 5-(2,6-di-morpholin-4-yl-pyrimidin-4-yl)-4-trifluoromethyl-pyridin-2-ylamine is described in WO07/084786 as Example 10.

In the context of the present invention, and as demonstrated in the examples, the PI3K inhibitor can be replaced by an inhibitor of the mammalian target of rapamycin (mTOR). Hence, as used herein, the terms “PI3K inhibitor” and “phosphoinositide 3-kinase (PI3K) inhibitor” compound also include mTOR inhibitors. In addition, as used herein, the terms “PI3K inhibitor” and “phosphoinositide 3-kinase (PI3K) inhibitor” also encompass inhibitors of other PI3K pathway components such as AKT. A mTOR inhibitor is a compound that decreases the activity of the target of rapamycin (mTOR) pathway. A decrease in activity of the target of rapamycin pathway is defined by a reduction of a biological function of the target of rapamycin. A target of rapamycin biological function includes for example, inhibition of the response to interleukin-2 (IL-2), blocking the activation of T- and B-cells, control of proliferation, and control of cell growth. A mTOR inhibitor acts for example by binding to protein FK-binding protein 12 (FKBP 12). mTOR inhibitors are known in the art or are identified using methods described herein. The m-TOR inhibitor is for example a macrolide antibiotic such as rapamycin, temsirolimus (2,2-bis(hydroxymethyl)propionic acid; CCI-779) or everolimus (RAD001); AP23573 or mimetics or derivatives thereof. Further mTOR inhibitors are temsirolimus, ridaforolimus (also known as AP23573), MK-8669 (formerly known as Deforolimus), sirolimus, zotarolimus and biolimus. Mimetics and derivatives of rapamycin are known in the art such as those describes in U.S. Pat. Nos. RE37.421; 5,985,890; 5,912,253; 5,728,710; 5,712,129; 5,648,361; 7,332,601; 7,282,505; 6,680,330. Thus, as used herein, the term PI3K inhibitor also includes mTOR inhibitors and/or compounds which inhibit both PI3K and mTOR, e.g. Compound A.

Janus kinases (JAKs) form a family of intracellular protein tyrosine kinases with four members, JAK1, JAK2, JAK3 and TYK2. These kinases are important in the mediation of cytokine receptor signaling which induces various biological responses including cell proliferation, differentiation and cell survival. Knock-out experiments in mice have shown that JAKs are inter alia important in hematopoiesis. In addition, JAK2 was shown to be implicated in myeloproliferative diseases and cancers. JAK2 activation by chromosome re-arrangements and/or loss of negative JAK/STAT (STAT=signal transducing and activating factor(s)) pathway regulators has been observed in hematological malignancies as well as in certain solid tumors.

Janus kinase 2 (commonly called JAK2) is a human protein that has been implicated in signaling by members of the type II cytokine receptor family (e.g. interferon receptors), the GM-CSF receptor family (IL-3R, IL-5R and GM-CSF-R), the gp130 receptor family (e.g. IL-6R), and the single chain receptors (e.g. Epo-R, Tpo-R, GH-R, PRL-R). JAK2 signaling is activated downstream from the prolactin receptor. JAK2 gene fusions with the TEL(ETV6) (TEL-JAK2) and PCM1 genes have been found in leukemia patients. Further, mutations in JAK2 have been implicated in polycythemia vera, essential thrombocythemia, and other myeloproliferative disorders. This mutation, a change of valine to phenylalanine at the 617 position, appears to render hematopoietic cells more sensitive to growth factors such as erythropoietin and thrombopoietin. Loss of Jak2 is lethal by embryonic day 12 in mice. JAK2 orthologs have been identified in all mammals for which complete genome data are available. The JAK-STAT signaling pathway transmits information from chemical signals outside the cell, through the cell membrane, and into gene promoters on the DNA in the cell nucleus, which causes DNA transcription and activity in the cell. The JAK-STAT system is a major signaling alternative to the second messenger system. The JAK-STAT system consists of three main components: a receptor, JAK and STAT. JAK is short for Janus Kinase, and STAT is short for Signal Transducer and Activator of Transcription. The receptor is activated by a signal from interferon, interleukin, growth factors, or other chemical messengers. This activates the kinase function of JAK, which autophosphorylates itself (phosphate groups act as “on” and “off” switches on proteins). The STAT protein then binds to the phosphorylated receptor. STAT is phosphorylated and translocates into the cell nucleus, where it binds to DNA and promotes transcription of genes responsive to STAT. In mammals, there are seven STAT genes, and each one binds to a different DNA sequence. STAT binds to a DNA sequence called a promoter, which controls the expression of other DNA sequences. This affects basic cell functions, like cell growth, differentiation and death. The JAK-STAT pathway is evolutionarily conserved, from slime molds and worms to mammals (but not fungi or plants). Disrupted or dysregulated JAK-STAT functionality (which is usually by inherited or acquired genetic defects) can result in immune deficiency syndromes and cancers.

JAKs, which have tyrosine kinase activity, bind to some cell surface cytokine and hormone receptors. The binding of the ligand to the receptor triggers activation of JAKs. With increased kinase activity, they phosphorylate tyrosine residues on the receptor and create sites for interaction with proteins that contain phosphotyrosine-binding SH2 domains. STATs possessing SH2 domains capable of binding these phosphotyrosine residues are recruited to the receptors, and are themselves tyrosine-phosphorylated by JAKs. These phosphotyrosines then act as binding sites for SH2 domains of other STATs, mediating their dimerization. Different STATs form hetero- or homodimers. Activated STAT dimers accumulate in the cell nucleus and activate transcription of their target genes. STATs may also be tyrosine-phosphorylated directly by receptor tyrosine kinases, such as the epidermal growth factor receptor, as well as by non-receptor tyrosine kinases such as c-src. The pathway is negatively regulated on multiple levels. Protein tyrosine phosphatases remove phosphates from cytokine receptors and activated STATs. Other suppressors of cytokine signalling (SOCS) inhibit STAT phosphorylation by binding and inhibiting JAKs or competing with STATs for phosphotyrosine binding sites on cytokine receptors. STATs are also negatively regulated by protein inhibitors of activated STAT (PIAS), which act in the nucleus through several mechanisms. For example, PIAS1 and PIAS3 inhibit transcriptional activation by STAT1 and STAT3 respectively by binding and blocking access to the DNA sequences they recognize.

Janus kinase inhibitor is a class of medicines that function by inhibiting the effect of one or more of the Janus kinase family of enzymes (JAK1, JAK2, JAK3, TYK2), interfering with the JAK-STAT signaling pathway.

Some JAK2 inhibitors are under development for the treatment of polycythemia vera, essential thrombocythemia, and myeloid metaplasia with myelofibrosis. Some inhibitors of JAK2 are in clinical trials, e.g. for psoriasis.

Examples of JAK2 inhibitors are: Lestaurtinib against JAK2, for acute myelogenous leukemia (AML), Ruxolitinib against JAK1/JAK2 for psoriasis, myelofibrosis, and rheumatoid arthritis, SB1518 against JAK2 for relapsed lymphoma, advanced myeloid malignancies, myelofibrosis and CIMF, CYT387 against JAK2 for myeloproliferative disorders, LY3009104 (INCB28050) against JAK1/JAK2 starting phase IIb for rheumatoid arthritis, INC424 (also known as INCB01842) against JAK2, COMPOUND D against JAK2, TG101348 against JAK2; for which phase I results for myelofibrosis have been published, LY2784544 against JAK2, BMS-911543 against JAK2, and NS-018 (Nakaya et al., 2011, Blood Cancer Journal, 1, e29; doi:10.1038/bcj.2011.29).

WO 2005/080393 discloses inter alia 7H-pyrrolo[2,3d]pyrimidin-2yl-amino derivatives which are useful in the treatment of disorders associated with abnormal or deregulated kinase activity.

Bioorganic & Medical Chemistry Letters 16 (2006), 2689 discloses design and synthesis of certain 7H-pyrrolo[2,3d]pyrimidines as focal adhesion kinase inhibitors.

As disclosed in WO2009/098236, it has been found that the 7-phenyl-7H-pyrrolo[2,3d]pyrimidin-2yl-amino derivatives of the formula III given below, have advantageous pharmacological properties and inhibit, for example, the tyrosine kinase activity of Janus kinases, such as JAK2 kinase and/or JAK3- (but also JAK-1-) kinase. Hence, the compounds of formula III are suitable, for example, to be used in the combination of the present invention for the treatment of diseases depending on the tyrosine kinase activity of JAK2 (and/or JAK3) kinase, especially proliferative diseases such as tumor diseases, leukaemias, polycythemia vera, essential thrombocythemia, and myelofibrosis with myeloid metaplasia.

In an aspect, the invention relates to compounds of the formula III,

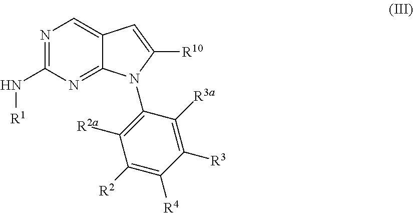

wherein
R¹ represents unsubstituted or substituted heterocyclyl, unsubstituted or substituted aryl, unsubstituted or substituted cycloalkyl;
R² represents hydrogen, halogen, lower alkyl, lower alkyloxy, lower haloalkyl, cycloalkyl, cycloalkyloxy, halocycloalkyl, cycloalkyloxy, halocycloalkyloxy;
R³ represents hydrogen, halogen, lower alkyl, lower alkyloxy, lower haloalkyl, cycloalkyl, cycloalkyloxy, halocycloalkyl, cycloalkyloxy, halocycloalkyloxy;
or R² and/or R³ are connected to R⁵ or R⁷ to form a cyclic moiety fused to the phenyl ring to which R²/R³ are attached;
R^{2a} represents hydrogen, halogen, lower alkyl, lower alkyloxy, lower haloalkyl, cycloalkyl, cycloalkyloxy, halocycloalkyl, cycloalkyloxy, halocycloalkyloxy;
R^{3a} represents hydrogen, halogen, lower alkyl, lower alkyloxy, lower haloalkyl, cycloalkyl, cycloalkyloxy, halocycloalkyl, cycloalkyloxy, halocycloalkyloxy;
R⁴ represents a group:

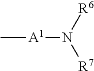

wherein A^{l} represents one of the following groups:

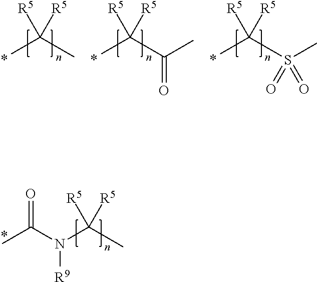

in which the atom marked * is bond to the phenyl ring;
   or
R⁴ represents one of the following groups:

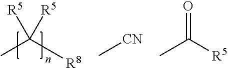

R⁵ represents independent from each other hydrogen, lower alkyl, lower haloalkyl, cycloalkyl, halocycloalkyl or form, together with the carbon to which they are attached a cycloalkyl;
R⁶ and R⁷ represent together with the nitrogen to which they are attached an optionally substituted heterocycle
   or
R⁶ represents hydrogen or optionally substituted alkyl and
R⁷ represents optionally substituted alkyl;
R⁸ represents alkyl, hydroxy, lower alkyloxy, lower haloalkyloxy, cycloalkyloxy, halocycloalkyloxy, lower alkyl-sulfonyl, lower-haloalkyl-sulfonyl, cycloalkyl-sulfonyl, halocycloalkyl-sulfonyl, lower alkyl-sulfinyl, lower haloalkyl-sulfinyl, cycloalkyl-sulfinyl, halocycloalkyl-sulfinyl;
R⁹ represents H or lower alkyl;
R¹⁰ represents hydrogen, lower alkyl, lower haloalkyl, cycloalkyl, halocycloalkyl;
n represents 0, 1 or 2;
   or salts thereof.

The radicals and symbols as used in the definition of a compound of formula IV have the meanings as disclosed in WO2009/098236 which publication is hereby incorporated into the present application by reference.

WO2008/148867 discloses quinoxaline compounds of the formula (IV)

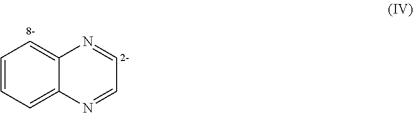

in which the 2- and 8-positions of the quinoxaline ring are substituted by cyclic groups. The compounds may be useful as inhibitors of the tyrosine kinase activity of Janus kinases, including JAK-2 and JAK-3 kinases. Example 98 of WO2008/148867 describes 8-(3,5-Difluoro-4-morpholin-4-ylmethyl-phenyl)-2-(1-piperidin-4-yl-1H-pyrazol-4-yl)-quinoxaline (COMPOUND D, also known as BSK805 or BSK-805)

The radicals and symbols as used in the definition of a compound of formula IV have the meanings as disclosed in WO2008/148867 which publication is hereby incorporated into the present application by reference.

As used herein, STAT5 refers to two highly related proteins, STAT5A and STAT5B, which are encoded by separate genes, but are 90% identical at the amino acid level (Grimley P M, Dong F, Rui H, 1999, Cytokine Growth Factor Rev. 10(2):131-157). Signal transducer and activator of transcription 5A (STAT5A) is a protein that in humans is encoded by the STAT5A gene. STAT5A orthologs have been identified in several placentals for which complete genome data are available. The protein encoded by this gene is a member of the STAT family of transcription factors. In response to cytokines and growth factors, STAT family members are phosphorylated by the receptor associated kinases, and then form homo- or heterodimers that translocate to the cell nucleus where they act as transcription activators. This protein is activated by, and mediates the responses of many cell ligands, such as IL2, IL3, IL7 GM-CSF, erythropoietin, thrombopoietin, and different growth hormones. Activation of this protein in myeloma and lymphoma associated with a TEL/JAK2 gene fusion is independent of cell stimulus and has been shown to be essential for the tumorigenesis. The mouse counterpart of this gene is found to induce the expression of BCL2L1/BCL-X(L), which suggests the antiapoptotic function of this gene in cells. STAT5A has been shown to interact with CRKL, Epidermal growth factor receptor, ERBB4, Erythropoietin receptor, Janus kinase 1, Janus kinase 2, MAPK1, NMI, and PTPN11. Signal transducer and activator of transcription 5B is a protein that in humans is encoded by the STAT5B gene. STAT5B orthologs have been identified in most placentals for which complete genome data are available. The protein encoded by this gene is a member of the STAT family of transcription factors. This protein mediates the signal transduction triggered by various cell ligands, such as IL2, IL4, CSF1, and different growth hormones. It has been shown to be involved in diverse biological processes, such as TCR signaling, apoptosis, adult mammary gland development, and sexual dimorphism of liver gene expression. This gene was found to fuse to retinoic acid receptor-alpha (RARA) gene in a small subset of acute promyelocytic leukemias (APML). STAT5B has been shown to interact with PTPN11, Janus kinase 2, Janus kinase 1 and Glucocorticoid receptor. STAT5 inhibitors are known in the art, see e.g. Cumaraswamy et al., 2011, MedChemComm, DOI: 10.1039/c1md00175b. These include pimozide, N′-((4-Oxo-4H-chromen-3-yl)methylene)nicotinohydrazide (COMPOUND E), “IQDMA” (N′-(11H-indolo[3,2-c]quinolin-6-yl)-N2,N2-dimethylethane-1,2-diamine; (COMPOUND F), as well as compounds 12, 13 and 14 as described in Cumaraswamy et al., 2011, (MedChemComm, DOI: 10.1039/c1md00175b; COMPOUND G, H and I, respectively).

Hence, the present invention also pertains to a combination such as a combined preparation or a pharmaceutical composition which comprises (a) a phosphoinositide 3-kinase (PI3K) inhibitor compound and (b) a compound which modulates the Janus Kinase 2 (JAK2)-Signal Transducer and Activator of Transcription 5 (STAT5) pathway. More particularly, in a first embodiment, the present invention relates to a combination which comprises (a) a phosphoinositide 3-kinase (PI3K) inhibitor compound and (b) a JAK2 modulator.

The terms “combination” and “combined preparation” as used herein also define a “kit of parts” in the sense that the combination partners (a) and (b) as defined above can be dosed independently or by use of different fixed combinations with distinguished amounts of the combination partners (a) and (b), i.e. simultaneously or at different time points. The parts of the kit of parts can then, e.g., be administered simultaneously or chronologically staggered, that is at different time points and with equal or different time intervals for any part of the kit of parts. The ratio of the total amounts of the combination partner (a) to the combination partner (b) to be administered in the combined preparation can be varied, e.g. in order to cope with the needs of a patient sub-population to be treated or the needs of the single.

As shown in the examples, it has been found that combination therapy with a PI3K/mTOR inhibitor and a JAK2-STAT5 inhibitor results in unexpected improvement in the treatment of tumor diseases. When administered simultaneously, sequentially or separately, the PI3K/mTOR inhibitor and the JAK2-STAT5 inhibitor interact in a synergistic manner to reduce cell number and tumor growth as well as decrease the number of circulating tumor cells and metastasis. This unexpected synergy allows a reduction in the dose required of each compound, leading to a reduction in the side effects and enhancement of the clinical effectiveness of the compounds and treatment.

Determining a synergistic interaction between one or more components, the optimum range for the effect and absolute dose ranges of each component for the effect may be definitively measured by administration of the components over different w/w ratio ranges and doses to patients in need of treatment. For humans, the complexity and cost of carrying out clinical studies on patients renders impractical the use of this form of testing as a primary model for synergy. However, the observation of synergy in one species can be predictive of the effect in other species and animal models exist, as described herein, to measure a synergistic effect and the results of such studies can also be used to predict effective dose and plasma concentration ratio ranges and the absolute doses and plasma concentrations required in other species by the application of pharmacokinetic/pharmacodynamic methods. Established correlations between tumor models and effects seen in man suggest that synergy in animals may e.g. be demonstrated in the tumor models as described in the Examples below.

In one aspect the present invention provides a synergistic combination for human administration comprising (a) PI3K inhibitor compound and (b) a compound which modulates the JAK2-STAT5 pathway, or pharmaceutically acceptable salts or solvates thereof, in a combination range (w/w) which corresponds to the ranges observed in a tumor model, e.g. as described in the Examples below, used to identify a synergistic interaction. Suitably, the ratio range in humans corresponds to a non-human range selected from between 50:1 to 1:50 parts by weight, 50:1 to 1:20, 50:1 to 1:10, 50:1 to 1:1, 20:1 to 1:50, 20:1 to 1:20, 20:1 to 1:10, 20:1 to 1:1, 10:1 to 1:50, 10:1 to 1:20, 10:1 to 1:10, 10:1 to 1:1, 1:1 to 1:50, 1.1 to 1:20 and 1:1 to 1:10. More suitably, the human range corresponds to a non-human range of the order of 10:1 to 1:1 or 5:1 to 1:1 or 2:1 to 1:1 parts by weight.

According to a further aspect, the present invention provides a synergistic combination for administration to humans comprising an (a) a PI3K inhibitor compound and (b) a compound which modulates the JAK2-STAT5 pathway or pharmaceutically acceptable salts thereof, where the dose range of each component corresponds to the synergistic ranges observed in a suitable tumor model, e.g. the tumor models described in the Examples below, primarily used to identify a synergistic interaction. Suitably, the dose range of the PI3K inhibitor compound in human corresponds to a dose range of 1-1000 mg/kg, for instance, 1-500 mg/kg, 1-1000 mg/kg 1-200 mg/kg, 1-100 mg/kg, 1-50 mg/kg, 1-30 mg/kg (e.g. 1-35 mg/kg or 1-10 mg/kg for Compound A, 1-25 mg/kg for Compound B) in a suitable tumor model, e.g. a mouse model as described in the Examples below.

For the compound which modulates the JAK2-STAT5 pathway, the dose range in the human suitably corresponds to a synergistic range of 1-50 mg/kg or 1-30 mg/kg (e.g. 1-25 mg/kg, 1-10 mg/kg or 1-2.5 mg/kg) in a suitable tumor model, e.g. a mouse model as described in the Examples below. Suitably, the dose of PI3K inhibitor compound for use in a human is in a range selected from 1-1200 mg, 1-500 mg, 1-100 mg, 1-50 mg, 1-25 mg, 500-1200 mg, 100-1200 mg, 100-500 mg, 50-1200 mg, 50-500 mg, or 50-100 mg, suitably 50-100 mg, once daily or twice daily (b.i.d.) or three times per day (t.i.d.), and the dose of compound which modulates the JAK2-STAT5 pathway is in a range selected from 1-1000 mg, 1-500 mg, 1-200 mg, 1-100 mg, 1-50 mg, 1-25 mg, 10-100 mg, 10-200 mg, 50-200 mg or 100-500 mg once daily, b.i.d or t.i.d.

In accordance with a further aspect the present invention provides a synergistic combination for administration to humans comprising an (a) a PI3K inhibitor compound at 10%-100%, preferably 50%-100% or more preferably 70%-100%, 80%-100% or 90%-100% of the maximal tolerable dose (MTD) and (b) a compound which modulates the JAK2-STAT5 pathway at 10%-100%, preferably 50%-100% or more preferably 70%-100%, 80%-100% or 90%-100% of the MTD. In an embodiment one of the compounds, preferably the PI3K inhibitor compound, is dosed at the MTD and the other compound, preferably the compound which modulates the JAK2-STAT5 pathway, is dosed at 50%-100% of the MTD, preferably at 60%-90% of the MTD. The MTD corresponds to the highest dose of a medicine that can be given without unacceptable side effects. It is within the art to determine the MTD. For instance the MTD can suitably be determined in a Phase I study including a dose escalation to characterize dose limiting toxicities and determination of biologically active tolerated dose level.

In one embodiment of the invention, (a) the phosphoinositide 3-kinase (PI3K) inhibitor compound inhibitor is selected from the group consisting of COMPOUND A, COMPOUND B or COMPOUND C.

In one embodiment of the invention, (b) the JAK2-STAT5 modulator is an inhibitor selected from the group consisting of Lestaurtinib, Ruxolitinib, SB1518, CYT387, LY3009104 (INCB28050), INC424 (also known as INCB01842), COMPOUND D (BSK-805), TG101348, LY2784544, BMS-911543 and NS-018.

Further aspects of the invention include kits and methods for predicting which subject will resist to BEZ based on the expression of IL-8 and/or JAK2/STAT5 in tumours of said subject or on the presence of IL-8 in the plasma of said subject.

The term “treating” or “treatment” as used herein comprises a treatment effecting a delay of progression of a disease. The term “delay of progression” as used herein means administration of the combination to patients being in a pre-stage or in an early phase of the proliferative disease to be treated, in which patients for example a pre-form of the corresponding disease is diagnosed or which patients are in a condition, e.g. during a medical treatment or a condition resulting from an accident, under which it is likely that a corresponding disease will develop.

The subject to be treated is usually a human. Although mostly referring to human, the present invention is however not limited to human. In the present invention, the subject can be any warm-blooded animal, including, next to human, but not limited to, animals such as cows, pigs, horses, chickens, cats, dogs, camels, etc.

In one embodiment of the present invention, the proliferative disease is breast cancer, in particular a metastatic breast cancer or a breast cancer of the triple negative type.

In another embodiment of the present invention, the proliferative disease is a solid tumor. The term “solid tumor” especially means breast cancer, ovarian cancer, cancer of the colon and generally the GI (gastro-intestinal) tract, cervix cancer, lung cancer, in particular small-cell lung cancer, and non-small-cell lung cancer, head and neck cancer, bladder cancer, cancer of the prostate or Kaposi's sarcoma. The present combination inhibits the growth of solid tumors, but also liquid tumors. Furthermore, depending on the tumor type and the particular combination used a decrease of the tumor volume can be obtained. The combinations disclosed herein are also suited to prevent the metastatic spread of tumors, e.g. of breast cancer, and the growth or development of micrometastases. The combinations disclosed herein are in particular suitable for the treatment of poor prognosis patients.

The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference.

It will be understood that references to the combination partners (a) and (b) are meant to also include the pharmaceutically acceptable salts. If these combination partners (a) and (b) have, for example, at least one basic center, they can form acid addition salts. Corresponding acid addition salts can also be formed having, if desired, an additionally present basic center. The combination partners (a) and (b) having an acid group (for example COOH) can also form salts with bases. The combination partner (a) or (b) or a pharmaceutically acceptable salt thereof may also be used in form of a hydrate or include other solvents used for crystallization.

A combination which comprises (a) a phosphoinositide 3-kinase inhibitor compound and (b) a compound which modulates the JAK2-STAT5 pathway, in which the active ingredients are present in each case in free form or in the form of a pharmaceutically acceptable salt and optionally at least one pharmaceutically acceptable carrier, will be referred to hereinafter as a COMBINATION OF THE INVENTION.

The COMBINATION OF THE INVENTION has both synergistic and additive advantages, both for efficacy and safety. Therapeutic effects of combinations of a phosphoinositide 3-kinase inhibitor compound with a compound which modulates the JAK2-STSAT5 pathway can result in lower safe dosages ranges of each component in the combination.

The pharmacological activity of a COMBINATION OF THE INVENTION may, for example, be demonstrated in a clinical study or in a test procedure as essentially described hereinafter. Suitable clinical studies are, for example, open label non-randomized, dose escalation studies in patients with advanced solid tumors. Such studies can prove the additive or synergism of the active ingredients of the COMBINATIONS OF THE INVENTION. The beneficial effects on proliferative diseases can be determined directly through the results of these studies or by changes in the study design which are known as such to a person skilled in the art. Such studies are, in particular, suitable to compare the effects of a monotherapy using the active ingredients and a COMBINATION OF THE INVENTION. Preferably, the combination partner (a) is administered with a fixed dose and the dose of the combination partner (b) is escalated until the Maximum Tolerated Dosage (MTD) is reached.

It is one objective of this invention to provide a pharmaceutical composition comprising a quantity, which is therapeutically effective against a proliferative disease comprising the COMBINATION OF THE INVENTION. In this composition, the combination partners (a) and (b) can be administered together, one after the other or separately in one combined unit dosage form or in two separate unit dosage forms. The unit dosage form may also be a fixed combination.

The pharmaceutical compositions according to the invention can be prepared in a manner known per se and are those suitable for enteral, such as oral or rectal, and parenteral administration to mammals (warm-blooded animals), including man. Alternatively, when the agents are administered separately, one can be an enteral formulation and the other can be administered parenterally.

The novel pharmaceutical composition contain, for example, from about 10% to about 100%, preferably from about 20% to about 60%, of the active ingredients. Pharmaceutical preparations for the combination therapy for enteral or parenteral administration are, for example, those in unit dosage forms, such as sugar-coated tablets, tablets, capsules or suppositories, and furthermore ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In preparing the compositions for oral dosage form, any of the usual pharmaceutical media may be employed, such as, for example, water, glycols, oils, alcohols, flavoring agents, preservatives, coloring agents; or carriers such as starches, sugars, microcristalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed.

In particular, a therapeutically effective amount of each of the combination partner of the COMBINATION OF THE INVENTION may be administered simultaneously or sequentially and in any order, and the components may be administered separately or as a fixed combination. For example, the method of delay of progression or treatment of a proliferative disease according to the invention may comprise (i) administration of the first combination partner in free or pharmaceutically acceptable salt form and (ii) administration of the second combination partner in free or pharmaceutically acceptable salt form, simultaneously or sequentially in any order, in jointly therapeutically effective amounts, preferably in synergistically effective amounts. The individual combination partners of the COMBINATION OF THE INVENTION can be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimes of simultaneous or alternating treatment and the term “administering” is to be interpreted accordingly.

The COMBINATION OF THE INVENTION can be a combined preparation or a pharmaceutical composition.

Moreover, the present invention relates to a method of treating a warm-blooded animal having a proliferative disease comprising administering to the animal a COMBINATION OF THE INVENTION in a quantity which is therapeutically effective against said proliferative disease.

Furthermore, the present invention pertains to the use of a COMBINATION OF THE INVENTION for the treatment of a proliferative disease and for the preparation of a medicament for the treatment of a proliferative disease.

Moreover, the present invention provides a commercial package comprising as active ingredients COMBINATION OF THE INVENTION, together with instructions for simultaneous, separate or sequential use thereof in the delay of progression or treatment of a proliferative disease.

Embodiments of the invention are represented by combinations comprising
Lestaurtinib and one or more compound selected from the group consisting of COMPOUND A, COMPOUND B, COMPOUND C, rapamycin, temsirolimus, everolimus, temsirolimus, ridaforolimus, MK-8669 sirolimus, zotarolimus and biolimus.
Ruxolitinib and one or more compound selected from the group consisting of COMPOUND A, COMPOUND B, COMPOUND C, rapamycin, temsirolimus, everolimus, temsirolimus, ridaforolimus, MK-8669 sirolimus, zotarolimus and biolimus.
SB1518 and one or more compound selected from the group consisting of COMPOUND A, COMPOUND B, COMPOUND C, rapamycin, temsirolimus, everolimus, temsirolimus, ridaforolimus, MK-8669 sirolimus, zotarolimus and biolimus.
CYT387 and one or more compound selected from the group consisting of COMPOUND A, COMPOUND B, COMPOUND C, rapamycin, temsirolimus, everolimus, temsirolimus, ridaforolimus, MK-8669 sirolimus, zotarolimus and biolimus.
LY3009104 and one or more compound selected from the group consisting of COMPOUND A, COMPOUND B, COMPOUND C, rapamycin, temsirolimus, everolimus, temsirolimus, ridaforolimus, MK-8669 sirolimus, zotarolimus and biolimus.
INC424 and one or more compound selected from the group consisting of COMPOUND A, COMPOUND B, COMPOUND C, rapamycin, temsirolimus, everolimus, temsirolimus, ridaforolimus, MK-8669 sirolimus, zotarolimus and biolimus.
COMPOUND D and one or more compound selected from the group consisting of COMPOUND A, COMPOUND B, COMPOUND C, rapamycin, temsirolimus, everolimus, temsirolimus, ridaforolimus, MK-8669 sirolimus, zotarolimus and biolimus.
TG101348 and one or more compound selected from the group consisting of COMPOUND A, COMPOUND B, COMPOUND C, rapamycin, temsirolimus, everolimus, temsirolimus, ridaforolimus, MK-8669 sirolimus, zotarolimus and biolimus.
LY2784544 and one or more compound selected from the group consisting of COMPOUND A, COMPOUND B, COMPOUND C, rapamycin, temsirolimus, everolimus, temsirolimus, ridaforolimus, MK-8669 sirolimus, zotarolimus and biolimus.
BMS-911543 and one or more compound selected from the group consisting of COMPOUND A, COMPOUND B, COMPOUND C, rapamycin, temsirolimus, everolimus, temsirolimus, ridaforolimus, MK-8669 sirolimus, zotarolimus and biolimus.
NS-018 and one or more compound selected from the group consisting of COMPOUND A, COMPOUND B, COMPOUND C, rapamycin, temsirolimus, everolimus, temsirolimus, ridaforolimus, MK-8669 sirolimus, zotarolimus and biolimus.
COMPOUND E and one or more compound selected from the group consisting of COMPOUND A, COMPOUND B, COMPOUND C, rapamycin, temsirolimus, everolimus, temsirolimus, ridaforolimus, MK-8669 sirolimus, zotarolimus and biolimus.
COMPOUND F and one or more compound selected from the group consisting of COMPOUND A, COMPOUND B, COMPOUND C, rapamycin, temsirolimus, everolimus, temsirolimus, ridaforolimus, MK-8669 sirolimus, zotarolimus and biolimus.
COMPOUND G and one or more compound selected from the group consisting of COMPOUND A, COMPOUND B, COMPOUND C, rapamycin, temsirolimus, everolimus, temsirolimus, ridaforolimus, MK-8669 sirolimus, zotarolimus and biolimus.
COMPOUND H and one or more compound selected from the group consisting of COMPOUND A, COMPOUND B, COMPOUND C, rapamycin, temsirolimus, everolimus, temsirolimus, ridaforolimus, MK-8669 sirolimus, zotarolimus and biolimus.
COMPOUND I and one or more compound selected from the group consisting of COMPOUND A, COMPOUND B, COMPOUND C, rapamycin, temsirolimus, everolimus, temsirolimus, ridaforolimus, MK-8669 sirolimus, zotarolimus and biolimus.

In another embodiment, the invention provides combinations comprising
COMPOUND A and one or more compound selected from the group consisting of Lestaurtinib, Ruxolitinib, SB1518, CYT387, LY3009104, INC424, LY2784544, BMS-911543, NS-018, COMPOUND D, TG101348, COMPOUND E, COMPOUND F, COMPOUND G, COMPOUND H and COMPOUND I.
COMPOUND B and one or more compound selected from the group consisting of Lestaurtinib, Ruxolitinib, SB1518, CYT387, LY3009104, INC424, LY2784544, BMS-911543, NS-018, COMPOUND D, TG101348, COMPOUND E, COMPOUND F, COMPOUND G, COMPOUND H and COMPOUND I.
COMPOUND C and one or more compound selected from the group consisting of Lestaurtinib, Ruxolitinib, SB1518, CYT387, LY3009104, INC424, LY2784544, BMS-911543, NS-018, COMPOUND D, TG101348, COMPOUND E, COMPOUND F, COMPOUND G, COMPOUND H and COMPOUND I.
Rapamycin and one or more compound selected from the group consisting of Lestaurtinib, Ruxolitinib, SB1518, CYT387, LY3009104, INC424, LY2784544, BMS-911543, NS-018, COMPOUND D, TG101348, COMPOUND E, COMPOUND F, COMPOUND G, COMPOUND H and COMPOUND I.
Temsirolimus and one or more compound selected from the group consisting of Lestaurtinib, Ruxolitinib, SB1518, CYT387, LY3009104, INC424, LY2784544, BMS-911543, NS-018, COMPOUND D, TG101348, COMPOUND E, COMPOUND F, COMPOUND G, COMPOUND H and COMPOUND I.
Everolimus and one or more compound selected from the group consisting of Lestaurtinib, Ruxolitinib, SB1518, CYT387, LY3009104, INC424, LY2784544, BMS-911543, NS-018, COMPOUND D, TG101348, COMPOUND E, COMPOUND F, COMPOUND G, COMPOUND H and COMPOUND I.
Temsirolimus and one or more compound selected from the group consisting of Lestaurtinib, Ruxolitinib, SB1518, CYT387, LY3009104, INC424, LY2784544, BMS-911543, NS-018, COMPOUND D, TG101348, COMPOUND E, COMPOUND F, COMPOUND G, COMPOUND H and COMPOUND I.
Ridaforolimus and one or more compound selected from the group consisting of Lestaurtinib, Ruxolitinib, SB1518, CYT387, LY3009104, INC424, LY2784544, BMS-911543, NS-018, COMPOUND D, TG101348, COMPOUND E, COMPOUND F, COMPOUND G, COMPOUND H and COMPOUND I.
MK-8669 and one or more compound selected from the group consisting of Lestaurtinib, Ruxolitinib, SB1518, CYT387, LY3009104, INC424, LY2784544, BMS-911543, NS-018, COMPOUND D, TG101348, COMPOUND E, COMPOUND F, COMPOUND G, COMPOUND H and COMPOUND I.
Sirolimus and one or more compound selected from the group consisting of Lestaurtinib, Ruxolitinib, SB1518, CYT387, LY3009104, INC424, LY2784544, BMS-911543, NS-018, COMPOUND D, TG101348, COMPOUND E, COMPOUND F, COMPOUND G, COMPOUND H and COMPOUND I.
Zotarolimus and one or more compound selected from the group consisting of Lestaurtinib, Ruxolitinib, SB1518, CYT387, LY3009104, INC424, LY2784544, BMS-911543, NS-018, COMPOUND D, TG101348, COMPOUND E, COMPOUND F, COMPOUND G, COMPOUND H and COMPOUND I.
Biolimus and one or more compound selected from the group consisting of Lestaurtinib, Ruxolitinib, SB1518, CYT387, LY3009104, INC424, LY2784544, BMS-911543, NS-018, COMPOUND D, TG101348, COMPOUND E, COMPOUND F, COMPOUND G, COMPOUND H and COMPOUND I.

In further aspects, the present inventions provides
a combination which comprises (a) a COMBINATION OF THE INVENTION, wherein the active ingredients are present in each case in free form or in the form of a pharmaceutically acceptable salt or any hydrate thereof, and optionally at least one pharmaceutically acceptable carrier; for simultaneous, separate or sequential use;
a pharmaceutical composition comprising a quantity which is jointly therapeutically effective against a proliferative disease of a COMBINATION OF THE INVENTION and at least one pharmaceutically acceptable carrier;
the use of a COMBINATION OF THE INVENTION for the treatment of a proliferative disease;
the use of a COMBINATION OF THE INVENTION for the preparation of a medicament for the treatment of a proliferative disease;
the use of a combination COMBINATION OF THE INVENTION wherein the PI3K inhibitor is selected from COMPOUND A, COMPOUND B, COMPOUND C, rapamycin, temsirolimus, everolimus, temsirolimus, ridaforolimus, MK-8669 sirolimus, zotarolimus and biolimus; and
the use of a COMBINATION OF THE INVENTION wherein the compound which modulates the JAK2-STAT5 pathway is a compound which inhibits JAK2, e.g. Lestaurtinib, Ruxolitinib, SB1518, CYT387, LY3009104 (INCB28050), INC424 (also known as INCB01842), LY2784544, BMS-911543, NS-018, or TG101348.

Moreover, in particular, the present invention relates to a combined preparation, which comprises (a) one or more unit dosage forms of a phosphoinositide 3-kinase inhibitor compound and (b) a compound which modulates the JAK2-STAT5 pathway.

Furthermore, in particular, the present invention pertains to the use of a combination comprising (a) a phosphoinositide 3-kinase inhibitor compound and (b) a compound which modulates the JAK2-STAT5 pathway for the preparation of a medicament for the treatment of a proliferative disease.

The effective dosage of each of the combination partners employed in the COMBINATION OF THE INVENTION may vary depending on the particular compound or pharmaceutical composition employed, the mode of administration, the condition being treated, the severity of the condition being treated. Thus, the dosage regimen the COMBINATION OF THE INVENTION is selected in accordance with a variety of factors including the route of administration and the renal and hepatic function of the patient. A physician, clinician or veterinarian of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics of the active ingredients' availability to target sites.

When the combination partners employed in the COMBINATION OF THE INVENTION are applied in the form as marketed as single drugs, their dosage and mode of administration can take place in accordance with the information provided on the package insert of the respective marketed drug in order to result in the beneficial effect described herein, if not mentioned herein otherwise.

COMPOUND A may be administered to a human in a dosage range varying from about 50 to 1000 mg/day. COMPOUND B may be administered to a human in a dosage range varying from about 25 to 800 mg/day. COMPOUND C may be administered to a human in a dosage range varying from about 25 to 800 mg/day.

As demonstrated in the examples, the term “compound” as used herein also includes siRNA decreasing or silencing the expression of a target gene. “RNAi” is the process of sequence specific post-transcriptional gene silencing in animals and plants. It uses small interfering RNA molecules (siRNA) that are double-stranded and homologous in sequence to the silenced (target) gene. Hence, sequence specific binding of the siRNA molecule with mRNAs produced by transcription of the target gene allows very specific targeted knockdown' of gene expression. “siRNA” or “small-interfering ribonucleic acid” according to the invention has the meanings known in the art, including the following aspects. The siRNA consists of two strands of ribonucleotides which hybridize along a complementary region under physiological conditions. The strands are normally separate. Because of the two strands have separate roles in a cell, one strand is called the “anti-sense” strand, also known as the “guide” sequence, and is used in the functioning RISC complex to guide it to the correct mRNA for cleavage. This use of “anti-sense”, because it relates to an RNA compound, is different from the antisense target DNA compounds referred to elsewhere in this specification. The other strand is known as the “anti-guide” sequence and because it contains the same sequence of nucleotides as the target sequence, it is also known as the sense strand. The strands may be joined by a molecular linker in certain embodiments. The individual ribonucleotides may be unmodified naturally occurring ribonucleotides, unmodified naturally occurring deoxyribonucleotides or they may be chemically modified or synthetic as described elsewhere herein.

In some embodiments, the siRNA molecule is substantially identical with at least a region of the coding sequence of the target gene to enable down-regulation of the gene. In some embodiments, the degree of identity between the sequence of the siRNA molecule and the targeted region of the gene is at least 60% sequence identity, in some embodiments at least 75% sequence identity, for instance at least 85% identity, 90% identity, at least 95% identity, at least 97%, or at least 99% identity.

Calculation of percentage identities between different amino acid/polypeptide/nucleic acid sequences may be carried out as follows. A multiple alignment is first generated by the ClustalX program (pairwise parameters: gap opening 10.0, gap extension 0.1, protein matrix Gonnet 250, DNA matrix IUB; multiple parameters: gap opening 10.0, gap extension 0.2, delay divergent sequences 30%, DNA transition weight 0.5, negative matrix off, protein matrix gonnet series, DNA weight IUB; Protein gap parameters, residue-specific penalties on, hydrophilic penalties on, hydrophilic residues GPSNDQERK, gap separation distance 4, end gap separation off). The percentage identity is then calculated from the multiple alignment as (N/T)*100, where N is the number of positions at which the two sequences share an identical residue, and T is the total number of positions compared. Alternatively, percentage identity can be calculated as (N/S)*100 where S is the length of the shorter sequence being compared. The amino acid/polypeptide/nucleic acid sequences may be synthesised de novo, or may be native amino acid/polypeptide/nucleic acid sequence, or a derivative thereof. A substantially similar nucleotide sequence will be encoded by a sequence which hybridizes to any of the nucleic acid sequences referred to herein or their complements under stringent conditions. By stringent conditions, we mean the nucleotide hybridises to filter-bound DNA or RNA in 6× sodium chloride/sodium citrate (SSC) at approximately 45° C. followed by at least one wash in 0.2×SSC/0.1% SDS at approximately 5-65° C. Alternatively, a substantially similar polypeptide may differ by at least 1, but less than 5, 10, 20, 50 or 100 amino acids from the peptide sequences according to the present invention Due to the degeneracy of the genetic code, it is clear that any nucleic acid sequence could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequences which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine; large non-polar, hydrophobic amino acids include phenylalanine, tryptophan and tyrosine; the polar neutral amino acids include serine, threonine, cysteine, asparagine and glutamine; the positively charged (basic) amino acids include lysine, arginine and histidine; and the negatively charged (acidic) amino acids include aspartic acid and glutamic acid. The accurate alignment of protein or DNA sequences is a complex process, which has been investigated in detail by a number of researchers. Of particular importance is the trade-off between optimal matching of sequences and the introduction of gaps to obtain such a match. In the case of proteins, the means by which matches are scored is also of significance. The family of PAM matrices (e.g., Dayhoff, M. et al., 1978, Atlas of protein sequence and structure, Natl. Biomed. Res. Found.) and BLOSUM matrices quantify the nature and likelihood of conservative substitutions and are used in multiple alignment algorithms, although other, equally applicable matrices will be known to those skilled in the art. The popular multiple alignment program ClustalW, and its windows version ClustalX (Thompson et al., 1994, Nucleic Acids Research, 22, 4673-4680; Thompson et al., 1997, Nucleic Acids Research, 24, 4876-4882) are efficient ways to generate multiple alignments of proteins and DNA. Frequently, automatically generated alignments require manual alignment, exploiting the trained user's knowledge of the protein family being studied, e.g., biological knowledge of key conserved sites. One such alignment editor programs is Align (http://www.gwdg.de/dhepper/download/; Hepperle, D., 2001: Multicolor Sequence Alignment Editor. Institute of Freshwater Ecology and Inland Fisheries, 16775 Stechlin, Germany), although others, such as JalView or Cinema are also suitable. Calculation of percentage identities between proteins occurs during the generation of multiple alignments by Clustal. However, these values need to be recalculated if the alignment has been manually improved, or for the deliberate comparison of two sequences. Programs that calculate this value for pairs of protein sequences within an alignment include PROTDIST within the PHYLIP phylogeny package (Felsenstein; http://evolution.gs.washington.edu/phylip.html) using the “Similarity Table” option as the model for amino acid substitution (P). For DNA/RNA, an identical option exists within the DNADIST program of PHYL1P. The dsRNA molecules in accordance with the present invention comprise a double-stranded region which is substantially identical to a region of the mRNA of the target gene. A region with 100% identity to the corresponding sequence of the target gene is suitable. This state is referred to as “fully complementary”. However, the region may also contain one, two or three mismatches as compared to the corresponding region of the target gene, depending on the length of the region of the mRNA that is targeted, and as such may be not fully complementary. In an embodiment, the RNA molecules of the present invention specifically target one given gene. In order to only target the desired mRNA, the siRNA reagent may have 100% homology to the target mRNA and at least 2 mismatched nucleotides to all other genes present in the cell or organism. Methods to analyze and identify siRNAs with sufficient sequence identity in order to effectively inhibit expression of a specific target sequence are known in the art. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group).

The length of the region of the siRNA complementary to the target, in accordance with the present invention, may be from 10 to 100 nucleotides, 12 to 25 nucleotides, 14 to 22 nucleotides or 15, 16, 17 or 18 nucleotides. Where there are mismatches to the corresponding target region, the length of the complementary region is generally required to be somewhat longer. In an embodiment, the inhibitor is a siRNA molecule and comprises between approximately 5 bp and 50 bp, in some embodiments, between 10 bp and 35 bp, or between 15 bp and 30 bp, for instance between 18 bp and 25 bp. In some embodiments, the siRNA molecule comprises more than 20 and less than 23 bp.

Because the siRNA may carry overhanging ends (which may or may not be complementary to the target), or additional nucleotides complementary to itself but not the target gene, the total length of each separate strand of siRNA may be 10 to 100 nucleotides, 15 to 49 nucleotides, 17 to 30 nucleotides or 19 to 25 nucleotides. The phrase “each strand is 49 nucleotides or less” means the total number of consecutive nucleotides in the strand, including all modified or unmodified nucleotides, but not including any chemical moieties which may be added to the 3′ or 5′ end of the strand. Short chemical moieties inserted into the strand are not counted, but a chemical linker designed to join two separate strands is not considered to create consecutive nucleotides.

The phrase “a 1 to 6 nucleotide overhang on at least one of the 5′ end or 3′ end” refers to the architecture of the complementary siRNA that forms from two separate strands under physiological conditions. If the terminal nucleotides are part of the double-stranded region of the siRNA, the siRNA is considered blunt ended. If one or more nucleotides are unpaired on an end, an overhang is created. The overhang length is measured by the number of overhanging nucleotides. The overhanging nucleotides can be either on the 5′ end or 3′ end of either strand.

The siRNA according to the present invention display a high in vivo stability and may be particularly suitable for oral delivery by including at least one modified nucleotide in at least one of the strands. Thus the siRNA according to the present invention contains at least one modified or non-natural ribonucleotide. A lengthy description of many known chemical modifications are set out in published PCT patent application WO 200370918. Suitable modifications for delivery include chemical modifications can be selected from among: a) a 3′ cap; b) a 5′ cap, c) a modified internucleoside linkage; or d) a modified sugar or base moiety. Suitable modifications include, but are not limited to modifications to the sugar moiety (i.e. the 2′ position of the sugar moiety, such as for instance 2′-O-(2-methoxyethyl) or 2′-MOE) (Martin et al., Helv. Chim. Acta, 1995, 78, 486-504) i.e., an alkoxyalkoxy group) or the base moiety (i.e. a non-natural or modified base which maintains ability to pair with another specific base in an alternate nucleotide chain). Other modifications include so-called ‘backbone’ modifications including, but not limited to, replacing the phosphoester group (connecting adjacent ribonucleotides) with for instance phosphorothioates, chiral phosphorothioates or phosphorodithioates. End modifications sometimes referred to herein as 3′ caps or 5′ caps may be of significance. Caps may consist of simply adding additional nucleotides, such as “T-T” which has been found to confer stability on a siRNA. Caps may consist of more complex chemistries which are known to those skilled in the art.

Design of a suitable siRNA molecule is a complicated process, and involves very carefully analysing the sequence of the target mRNA molecule. On exemplary method for the design of siRNA is illustrated in WO2005/059132. Then, using considerable inventive endeavour, the inventors have to choose a defined sequence of siRNA which has a certain composition of nucleotide bases, which would have the required affinity and also stability to cause the RNA interference. The siRNA molecule may be either synthesised de novo, or produced by a micro-organism. For example, the siRNA molecule may be produced by bacteria, for example, E. coli. Methods for the synthesis of siRNA, including siRNA containing at least one modified or non-natural ribonucleotides are well known and readily available to those of skill in the art. For example, a variety of synthetic chemistries are set out in published PCT patent applications WO2005021749 and WO200370918. The reaction may be carried out in solution or, in some embodiments, on solid phase or by using polymer supported reagents, followed by combining the synthesized RNA strands under conditions, wherein a siRNA molecule is formed, which is capable of mediating RNAi. It should be appreciated that siNAs (small interfering nucleic acids) may comprise uracil (siRNA) or thyrimidine (siDNA). Accordingly the nucleotides U and T, as referred to above, may be interchanged. However it is preferred that siRNA is used. For the avoidance of doubt, the term siRNA as used herein also includes miRNA, shRNA and shRNAmir.

Gene-silencing molecules, i.e. inhibitors, used according to the invention are in some embodiments, nucleic acids (e.g. siRNA or antisense or ribozymes). Such molecules may (but not necessarily) be ones, which become incorporated in the DNA of cells of the subject being treated. Undifferentiated cells may be stably transformed with the gene-silencing molecule leading to the production of genetically modified daughter cells (in which case regulation of expression in the subject may be required, e.g. with specific transcription factors, or gene activators). The gene-silencing molecule may be either synthesized de novo, and introduced in sufficient amounts to induce gene-silencing (e.g. by RNA interference) in the target cell. Alternatively, the molecule may be produced by a micro-organism, for example, E. coli, and then introduced in sufficient amounts to induce gene silencing in the target cell. The molecule may be produced by a vector harboring a nucleic acid that encodes the gene-silencing sequence. The vector may comprise elements capable of controlling and/or enhancing expression of the nucleic acid. The vector may be a recombinant vector. The vector may for example comprise plasmid, cosmid, phage, or virus DNA. In addition to, or instead of using the vector to synthesize the gene-silencing molecule, the vector may be used as a delivery system for transforming a target cell with the gene silencing sequence.

The recombinant vector may also include other functional elements. For instance, recombinant vectors can be designed such that the vector will autonomously replicate in the target cell. In this case, elements that induce nucleic acid replication may be required in the recombinant vector. Alternatively, the recombinant vector may be designed such that the vector and recombinant nucleic acid molecule integrates into the genome of a target cell. In this case nucleic acid sequences, which favor targeted integration (e.g. by homologous recombination) are desirable. Recombinant vectors may also have DNA coding for genes that may be used as selectable markers in the cloning process.

The recombinant vector may also comprise a promoter or regulator or enhancer to control expression of the nucleic acid as required. Tissue specific promoter/enhancer elements may be used to regulate expression of the nucleic acid in specific cell types, for example, endothelial cells. The promoter may be constitutive or inducible.

Alternatively, the gene silencing molecule may be administered to a target cell or tissue in a subject with or without it being incorporated in a vector. For instance, the molecule may be incorporated within a liposome or virus particle (e.g. a retrovirus, herpes virus, pox virus, vaccina virus, adenovirus, lentivirus and the like). Alternatively a “naked” siRNA or antisense molecule may be inserted into a subject's cells by a suitable means e.g. direct endocytotic uptake.

The gene silencing molecule may also be transferred to the cells of a subject to be treated by transfection, infection, microinjection, cell fusion, protoplast fusion or ballistic bombardment. For example, transfer may be by: ballistic transfection with coated gold particles; liposomes containing a siNA molecule; viral vectors comprising a gene silencing sequence or means of providing direct nucleic acid uptake (e.g. endocytosis) by application of the gene silencing molecule directly.

In an embodiment of the present invention siNA molecules may be delivered to a target cell (whether in a vector or “naked”) and may then rely upon the host cell to be replicated and thereby reach therapeutically effective levels. When this is the case the siNA is in some embodiments, incorporated in an expression cassette that will enable the siNA to be transcribed in the cell and then interfere with translation (by inducing destruction of the endogenous mRNA coding the targeted gene product).

The following Examples illustrate the invention described above; they are not, however, intended to limit the scope of the invention in any way. The beneficial effects of the COMBINATION OF THE INVENTION can also be determined by other test models known as such to the person skilled in the pertinent art.

### FIGURE LEGEND

FIG. 1. Dual PI3K/mTOR inhibition by COMPOUND A activates JAK2/STAT5 in vitro and in vivo (A) Immunoblots of lysates from time-course experiments performed in three different breast cancer lines treated with COMPOUND A (BEZ-235) as indicated (human lines: MDA 468 and MDA 231 LM2). For the in vivo data, SCID/beige mice bearing xenografts were treated once with vehicle or 30 mg/kg COMPOUND A before dissection at time points indicated. (B) Immunoblots of lysates from MDA 468 and MDA 231 LM2 human cell lines in which JAK2 and JAK1 were depleted by siRNA. siNT=non-target control siRNA. (C) Immunoblots of lysates from 8h-BEZ treated MDA 468 cells in which JAK2/STAT5 signalling was blocked by siRNA (left panel) or the JAK2-specific inhibitor BSK-805 (COMPOUND D) (right panel). pJAK2 was measured by ELISA and normalized to total JAK2 levels. Densitometric quantification is given for pSTAT5 normalized to total STATS.

FIG. 2. Combination of COMPOUND A with the JAK2 inhibitor COMPOUND D reduces cell viability and triggers apoptosis (A) Bar graph showing the mean percentage of cell viability as measured by the WST-1 survival assay of cell lines grown under low serum conditions (0.5%) and treated with 300 nM BEZ (Compound A) and/or 350 nM BSK (Compound D) for 72 h as (left panel). Immunoblots of lysates from the same cell lines after 8 h of treatment (right panel). Data are mean±SD of 4 independent experiments; *P<0.05, **P<0.01. (B) Immunoblots of lysates from the three cell lines after 20 h of single and combination treatment. (C) Bar graph showing the mean percentage of apoptotic cells after 48 h of treatment as measured by FACS analysis of AnnexinV and PI stained cells treated with 300 nM BEZ (Compound A) and/or 350 nM BSK (Compound D) as indicated (left). Immunoblots of lysates from cell treated with 300 nM BEZ (Compound A) and/or 350 nM BSK (Compound D) for 24 h (right). Data are mean±SD (n=5, *P<0.05, **P<0.01. (C) Bar graph showing the mean percentage of apoptotic cells after 48 h of treatment as measured by FACS analysis of AnnexinV and PI stained cells treated with 300 nM BEZ and/or 350 nM BSK as indicated (left). Immunoblots of lysates from cell treated with 300 nM BEZ and/or 350 nM BSK for 24 h (right). Data are mean±SD (n=5, *P<0.05, **P<0.01.

FIG. 3. Compound A (Dual PI3K/mTOR inhibition) induces IL-8 secretion in breast cancer (A) A soluble factor from BEZ-treated cells activates JAK2/STAT5. Shown are immunoblots of lysates of cells treated for 30 min with conditioned media from cells treated with 300 nM BEZ for 24 h. As a control for the BEZ present in the condition media, we used lysates of cells treated with medium containing BEZ (SN BEZ ctrl). (B) IL-8 is secreted upon treatment of breast cancer cells with BEZ. Cytokine arrays showing expression of the indicated cytokines in supernatant (upper panel) or tumor lysates (lower panel) of cells treated with 300 nM BEZ for 24 h or allografts bearing mice treated with 30 mg/kg BEZ for 10 days, respectively. Mouse MIP2 is the functional homologue of human IL-8. (C) Kinetics of IL-8 overexpression upon BEZ treatment. Bar graph showing time course of IL-8 secretion (left panel) and mRNA upregulation (right panel) in cells treated with 300 nM BEZ as indicated. Levels of IL-8 were measured by ELISA and RQ-PCR respectively and are shown as mean±SD (n=4, *P<0.05). (D) BEZ increased IL-8 secretion and phosphorylation of JAK2/STAT5 in a panel of breast cancer cell lines. Graph of the correlation (Coeff.=0.77) between IL-8 secretion and JAK2 activation in a panel of triple negative and luminal breast cancer cell lines treated for 8 h with 300 nM of BEZ-235 (see Table 1).

FIG. 4. Compound A (PI3K/mTOR inhibition) induces a biphasic activation of JAK2/STAT5 (A) lmmunoblots of lysates from cells treated with DMSO or 300 nM BEZ alone or in combination with IgG or CXCR1 blocking antibody added 30 min before lysis. (B) Immunoblots of lysates from cells treated with 300 nM of BEZ as indicated. (C) Immunoprecipitation (IP) and immunoblotting of lysates from cells treated with DMSO or 300 nM BEZ for 8 h. WCL: Whole cell lysates. (D) Immunoblots of lysates from cells in which IRS1 was depleted by siRNA before treatment with DMSO or 300 nM BEZ for 8 h. siNT refers to non-targeting siRNA. (E) Bar graph showing the levels of IL-8 secretion (left) and mRNA (right) upon treatment with 300 nM BEZ and/or 350 nM BSK for 20 h (left) or 8 h (left). Levels of IL-8 were measured by ELISA and RQ-PCR, respectively, and are shown as mean±SD (n=4, *P<0.05).

FIG. 5. Cotargeting PI3K/mTOR (compound A) and JAK2/STAT5 (compound D) reduces primary tumor growth and metastasis (A)-(D) Growth curves of tumors and immunoblots of tumor lysates of mice treated with vehicle control (VHC), 30 mg/kg BEZ, 120 mg/kg BSK or 25 mg/kg BEZ and 100 mg/kg BSK. In C, JAK2 is inhibited by dox administration leading to activation of the JAK2 shRNA (shJAK2). shNT refers to non-targeting shRNA, injection refers to orthotopic cell injection and the arrows indicate initiation of treatment and/or administration of dox. In B, shown are representative bioluminescent images of luciferase expressing MDA231 LM2 tumors one day before the end of the treatment. Immunoblotting was performed on tumors harvested after 14 days of treatment for MDA468, 10 days of treatment for MDA231 LM2 and 6 days of treatment for 4T-1. Results are presented as mean tumor volume±SEM (n=4-8, *P<0.05, **P<0.01, ***P<0.001). (E) Bar graph showing the number of circulating tumor cells (CTCs) as measured by FACS analysis of GFP+ cells in tail vein blood performed 21 days (MDA231 LM2) or 5 days (4T-1) after initiation of the treatment as in A-D. Data are expressed as GFP+ CTCs normalized to 105 peripheral blood cells (PBCs) and are the means±SEM (n=4). (F) Upper left and middle: Representative images of lungs harvested 4 weeks after removal of the primary tumors from tumor bearing mice treated as in B-C. Upper right: Representative images of lungs harvested from tumor bearing mice after 19 days of treatment as D. Bar graphs show the metastatic index of mice treated as in B-D. The metastatic index was calculated by dividing the total number of visible lung metastatic nodules by tumor volume. Results are presented as the means±SD (n=4-8, *P<0.05, **P<0.01).

FIG. 6. IL-8 secretion in vivo is enhanced upon compound A (BEZ) treatment and reduced by compound D (blockade of JAK2/STAT5). (A) Bar graphs showing IL-8 levels measured by ELISA (left and middle) or quantification of cytokine arrays (right) in tumors of mice treated as in FIG. 5A-D. Results are the means±SD (n=3-8). (B) Bar graphs showing IL-8 levels measured by ELISA in plasma of mice bearing tumor treated as in FIG. 5 A-C. Results are the means±SD (n=4). (C) Schematics illustrating the identified positive feedback loop triggered by inhibition of PI3K/mTOR and its blockade by JAK2/STAT5 inhibition.

FIG. 7. Compound A (BEZ treatment) activates JAK2/STAT5 and IL-8 secretion in human primary triple-negative breast tumors (A) Immunoblots of lysates from primary triple-negative breast tumors grown in immunodeficient mice and treated for 4 days with 30 mg/kg BEZ or vehicle (VHC). (B) Bar graphs showing IL-8 levels measured by ELISA in the dissected tumors from, or in the plasma of, mice at day 3 of treatment with 30 mg/kg BEZ or vehicle (VHC).

FIG. 8. Dual PI3K/mTOR inhibition as well as single MK or single mTOR inhibition activates JAK2/STAT5. (A) Immunoblots of cell lysates from time-course experiments with BEZ treatment as indicated. (B) Immunoblots of cell lysates from time-course experiments with RAD001 or BKM120 treatment as indicated.

FIG. 9. Combined PI3K/mTOR and JAK2/STAT5 inhibition reduce cell viability (A) FACS Cell Cycle analysis of the three breast cancer cell lines treated with 300 nM compound A (BEZ) and/or 350 nM compound D (BSK) for 48 h. Data are mean±SD (n=4, *P<0.05, **P<0.01). (B) and (C) Bar graphs showing WST-1 survival assays after 72 h of treatment with 300 nM compound A (BEZ) and/or 350 nM compound D (BSK) at full serum conditions (10% FCS) or treatment with compound A (BEZ) and doxycycline-inducible downregulation of JAK2 at low serum conditions (0.5% FCS). Immunoblots of cell lysates showing knock-down of JAK2 in both cell lines ((C), right panel).

FIG. 10. The IL-8 receptor CXCR1 is expressed on breast cancer cells and IL-8 activates JAK2/STAT5 (A) Bar graph showing FACS analysis of expression levels of the two IL-8 receptors CXCR1 and CXCR2 on MDA468 (upper panel) and MDA231 LM2 (lower panel) confirming the presence of CXCR1 surface receptor in the cells. Data are mean±SD (n=3, *P<0.05, **P<0.01). (B) Immunoblots of cells lysed 30min after stimulation with recombinant cytokines (IL-8, IL-6, G-CSF 10 ng/ml, EPO 20 Units/ml).

FIG. 11. Dual PI3K/mTOR and JAK/STAT5 inhibition reduce primary tumor growth and have no adverse effects on body weight of the mice (A) Body weight of MDA 468 tumor bearing mice was monitored weekly and no significant changes upon treatment/combination were observed (left panel). Weight of dissected tumors before processing (right panel). Data are the means±SEM of each n=6-8. (B) As a measure for proliferation, mitotic figures were assessed on H&E stained tumor slices and are shown as the mitosis index. Data are the means±SEM of each n=6-8 tumors/treatment group. (C) IHC stainings for pSTAT5, pAKT and pS6 were performed on the treated tumors, representative pictures are shown. (D) Body weight and weight of tumors in the MDA 231 LM2 model (see (A)). Data are the means±SD of each n=7-8. (E) and (F) Mitosis index of treated 4T-1 tumors at the end of treatment, see (B). Data are the means±SD of each n=5-7 tumors/treatment group. (G) IHC stainings for pSTAT5, pAKT and pS6 were performed on the treated tumors, representative pictures are shown.

FIG. 12. IL-8 and JAK2 signalling are higher in metastatic cells (A) Immunoblots and ELISA measurements of cell lysates from parental breast cancer lines (168FARN and MDA 231) versus their metastatic sublines (4T-1 and MDA 231 LM2) (B) Graphs showing IL-8 supernatant ELISA and IL-8 RQ-PCR in MDA231 and MDA231 LM2 cells. Results presented are the means±SD (n=3, *P<0.05). (C) Pictures of FACS analysis of CXCR1 and CXCR2 expression on MDA 231 and MDA 231 LM2 cells. Results shown are representative graphs of three independent experiments. (D) Bar graph showing end-point expression levels of the IL-8 receptor CXCR1 on treated tumors as measured by FACS, MFI=Mean fluorescence intensity. (E) Graph showing basal IL-8 secretion blotted against invasive potential of luminal (in grey) and triple negative cell lines (in black).

FIG. 13. Compound A (BEZ)-mediated JAK2 and IL-8 activation correlate with sensitivity towards the inhibitor. (E) BEZ insensitive breast cancer lines (Brachmann et al., 2009) display higher BEZ-induced JAK2 phosphorylation and IL-8 secretion than sensitive lines. Graph showing breast cancer lines as in Table 1 blotted based on levels of pJAK2 (left) and IL-8 secretion (right) upon BEZ treatment and sensitivity towards BEZ.

FIG. 14. Cell viability. Bar graphs showing the mean percentages of cell viability as measured by the WST-1 assay of two BEZ-insensitive lines (left panel) and two BEZ-sensitive lines (right panel) grown under 0.5% serum and treated with 300 nM BEZ and/or 350 nM BSK for 72 h. Data are means±SEM (n=4, *p<0.05).

FIG. 15. Co-targeting PI3K/mTOR and JAK2/STAT5 reduces primary tumor growth, tumor seeding and metastasis.(A) Drawings of the experimental setup. (B) Representative IHC pictures of lungs from VHC-, BEZ-, BSK- and BEZ/BSK-treated animals. Left panel: H&E- (left) and Vimentin- (right) stained lungs from MDA231 LM2-bearing animals, treated as described. Scale bar 250 μm. Right panel: H&E-stained lungs from 4T-1-bearing animals, treated as described. Arrows indicate metastases; the images to the right are magnifications of single metastatic foci. Scale bar 200 μm.

FIG. 16. (A) Bar graph showing the percentages of vimentin-positive lung area per section of mice treated as described. Results are presented as means±SEM (n=8). (B) BSK reduces metastasis in a tumor cell-autonomous manner. Left panel: drawing of the experimental setup. Mice bearing MDA231 LM2 shJAK2 or MDA231 LM2 shNT tumors were treated with BSK as described. Right panel: Bar graph showing the metastatic index calculated by dividing the total number of visible lung metastatic nodules by tumor volume. Results are presented as means±SEM (n=3-4, *p<0.05).

FIG. 17. (A) Bar graphs showing relative invasion of MDA231 LM2 cells seeded on Matrigel-coated Boyden chambers and treated with 300 nM BEZ, 350 nM BSK and/or CXCR1 blocking antibody. Invasion was assessed after 48 h. Data represent relative invasion values normalized to cell number and are means±SEM (n=4, *p<0.05). (B) Bar graph showing percentages of CXCR1⁺ cells in MDA231 LM2 tumors of mice treated as described. Data are means±SEM (n=4-6, *p<0.05). (C) Representative dot plot of FACS analyses performed on CXCR1- (upper panel), AnnexinV- and PI-stained (lower panel) MDA231 LM2 cells treated with inhibitors as described. Bar graphs showing the mean percentages of apoptotic and dead cells after 48 h of treatment. Data are means±SEM (n=4, *p<0.05). (D) Upper panel: drawing of the experimental setup. Mice bearing MDA231 LM2 tumors were treated as described. The tumors were dissected, dissociated and re-transplanted at different dilutions. Cell viability prior to re-transplantation was analyzed by PI-FACS staining and was found to be equal in all treatment groups (data not shown). Lower panel: Bar graph showing the TIC frequencies after treatment as described. Data are mean estimates from three independent experiments, total n=7 mice, *p<0.05, ***p<0.0001.

FIG. 18. BEZ235 treatment activates JAK2/STAT5 and IL-8 secretion in primary human TNBC xenografts. (A) Immunoblots of lysates from primary TNBC xenografts treated for 4 days with 30 mg/kg BEZ or VHC. ELISA data are means±SD (n=3). (B) Bar graphs showing IL-8 levels measured by ELISA in the dissected tumors from or in the plasma of mice at day 3 of treatment with 30 mg/kg BEZ or VHC. Data are means±SEM (n=3-4, *p<0.05).

FIG. 19. Co-targeting PI3K/mTOR and JAK2 increases event-free and overall survival in two models of metastatic breast cancer. (A) Upper panel: Drawing of the experimental setup. Lower panel: Kaplan-Meier survival curves of MDA231 LM2 (left) and 4T-1 (right) tumor-bearing mice treated with BEZ and/or BSK as described. An event was scored when a tumor reached 1 cm³ (n=4, *p<0.05, **p<0.01). (B) Upper panel: Drawing of the experimental setup. Lower panel: Kaplan-Meier survival curves of MDA231 LM2 (left) and 4T-1 (right) tumor-bearing mice treated with BEZ and/or BSK as described. An event was scored when a mouse showed any sign of distress; (n=4, *p<0.05, **p<0.01).

FIG. 20. Inhibition of CXCR1 blocks p-FAK and the first phase of JAK2/STAT5 activation is EGFR independent. (A) Bar graph showing levels of CXCR1 mRNA in MDA468 and MDA231 LM2 cells which were transfected with a non-targeting siRNA (siNT) and two different siRNAs targeting CXCR1. CXCR1 levels were measured by RT-qPCR and are shown as means±SEM (n=2).(B) Immunoblots of lysates from cells transiently transfected with a non-targeting siRNA (siNT) or an siRNA targeting CXCR1 (siCXCR) and treated with DMSO or 300 nM BEZ. ELISA data are means±SD (n=3). (C) Immunoblots of lysates from cells treated with 300 nM BEZ235 and/or 100 nM AEE788 for 8 h.

FIG. 21. JAK2/STAT5 and IL-8/CXCR1 signaling promote invasion and metastasis. (A) Representative pictures from the invasion assays performed with MDA231 LM2 cells treated with 300 nM BEZ235 and/or 350 nM BSK for 48 h. Scale bar 50 μm. (B) Table showing the take rates of tumors from mice treated as described. Estimates of tumor initiating cell (TIC) frequency and confidence intervals were calculated using R and the “statmod” package (Hu and Smyth, 2009).

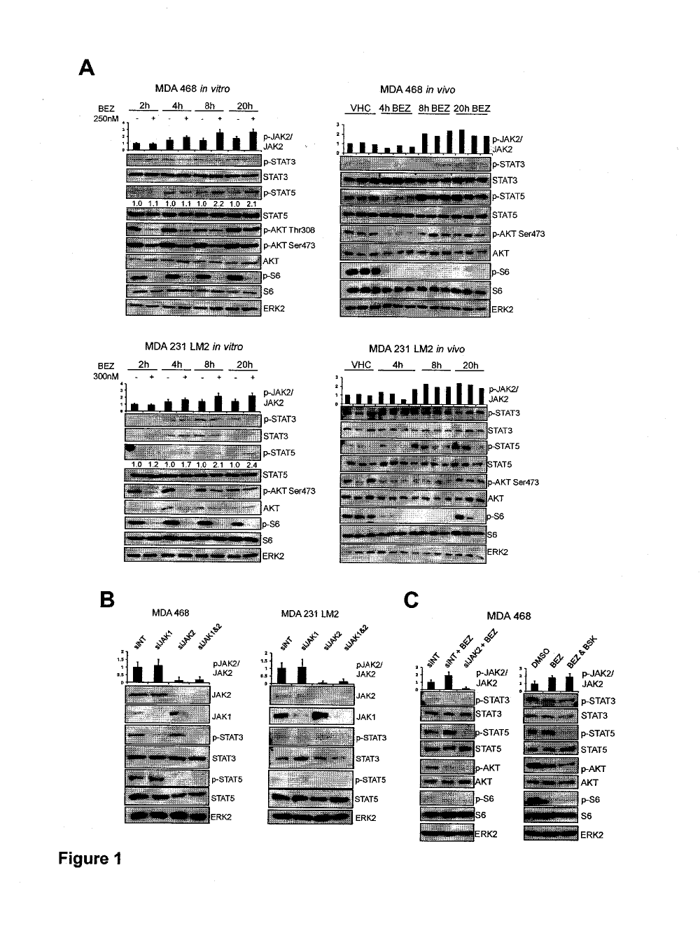

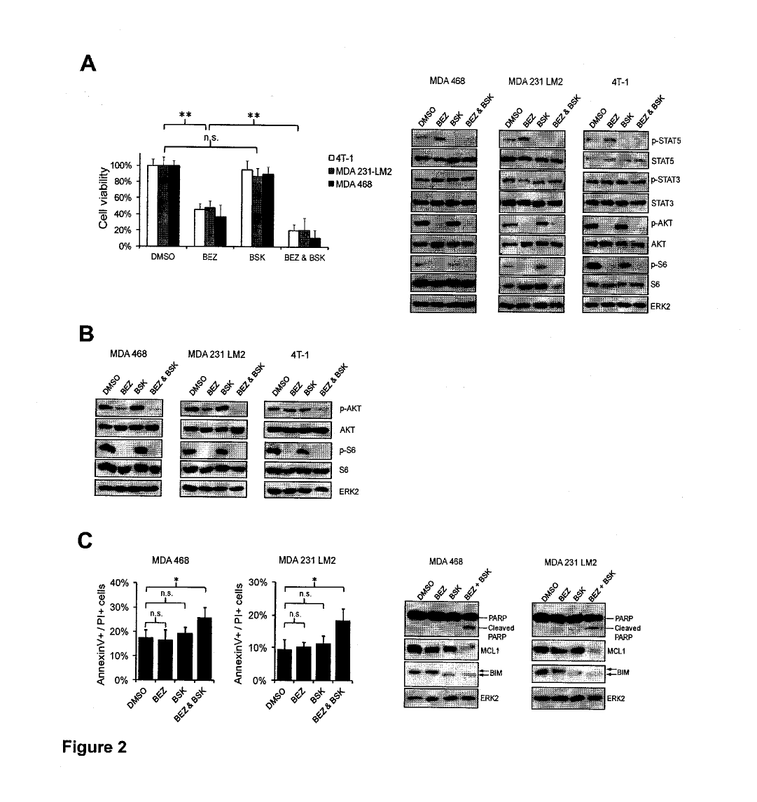

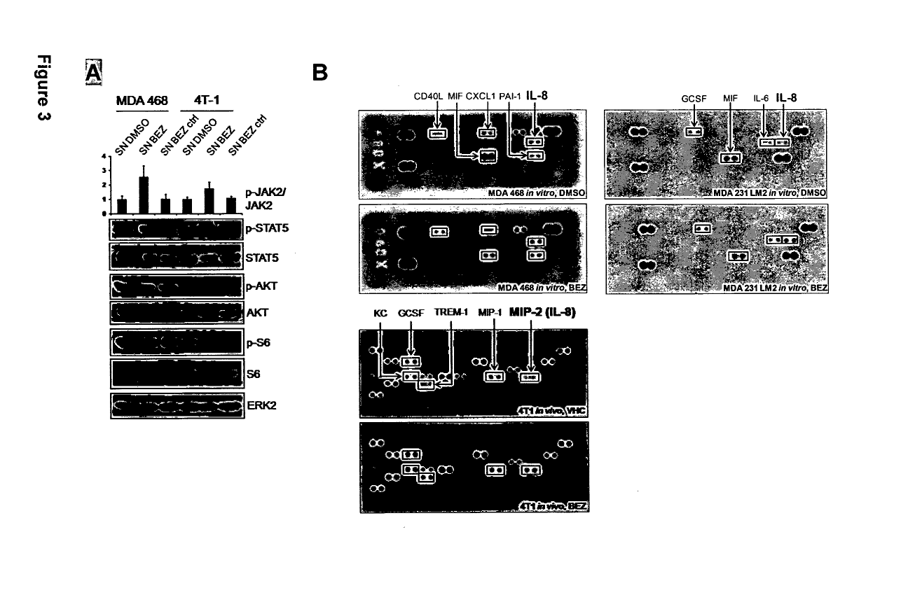

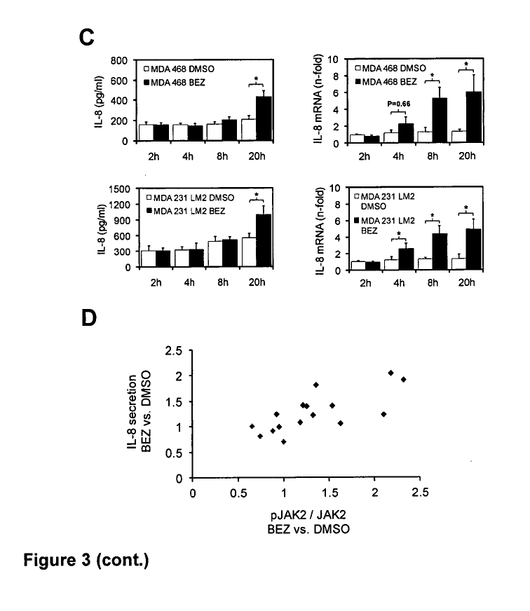

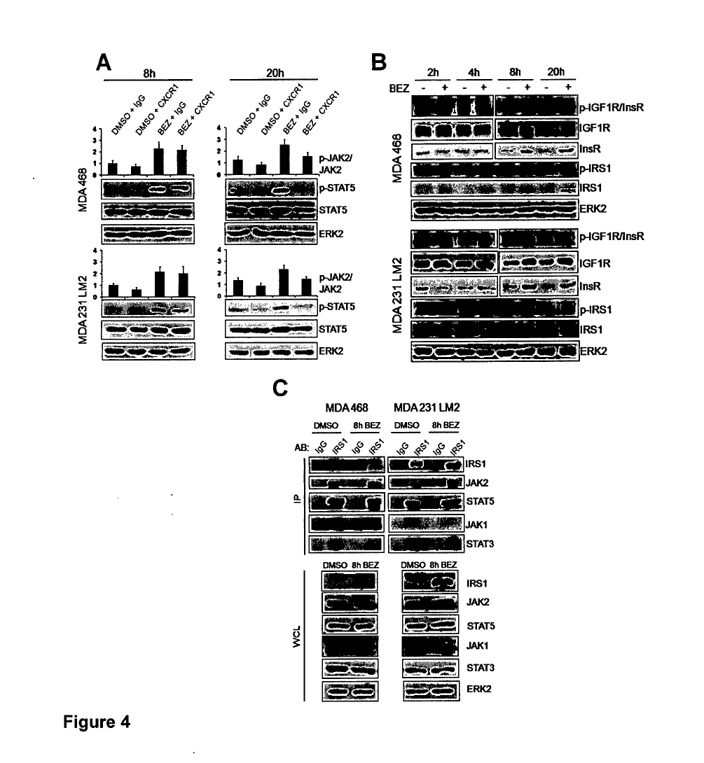

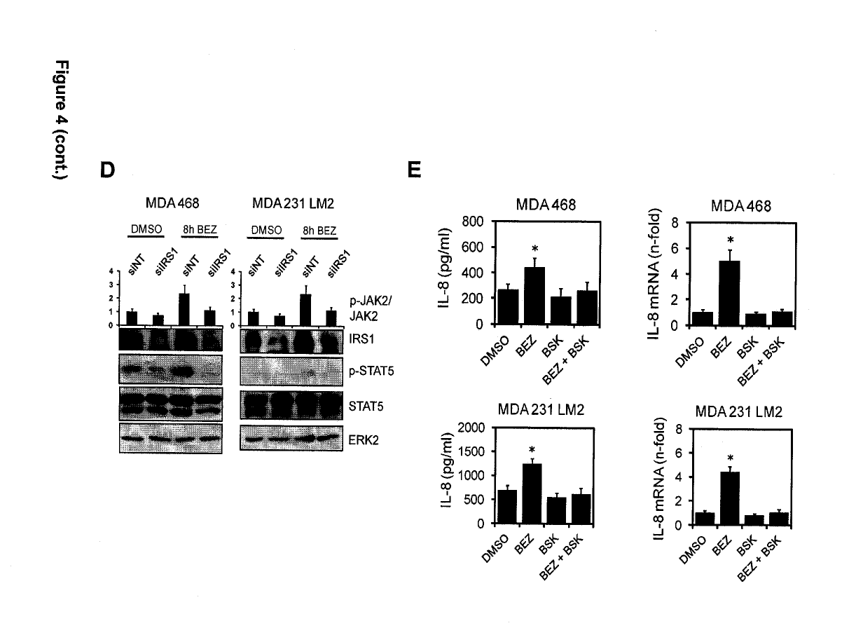

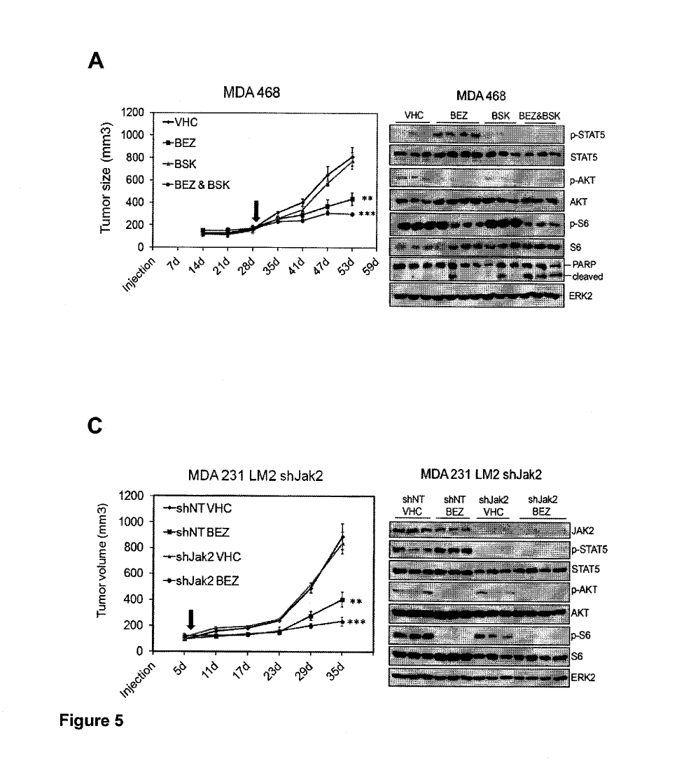

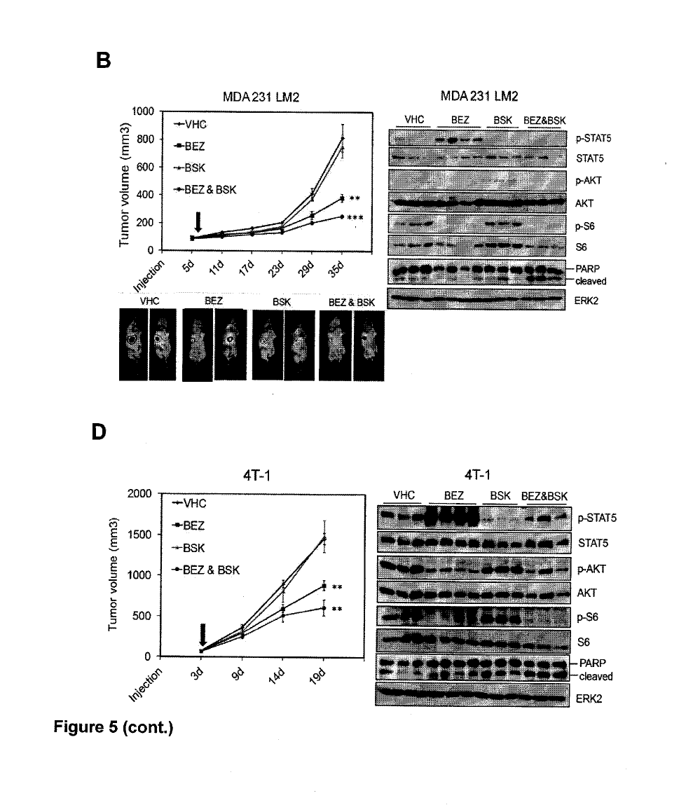

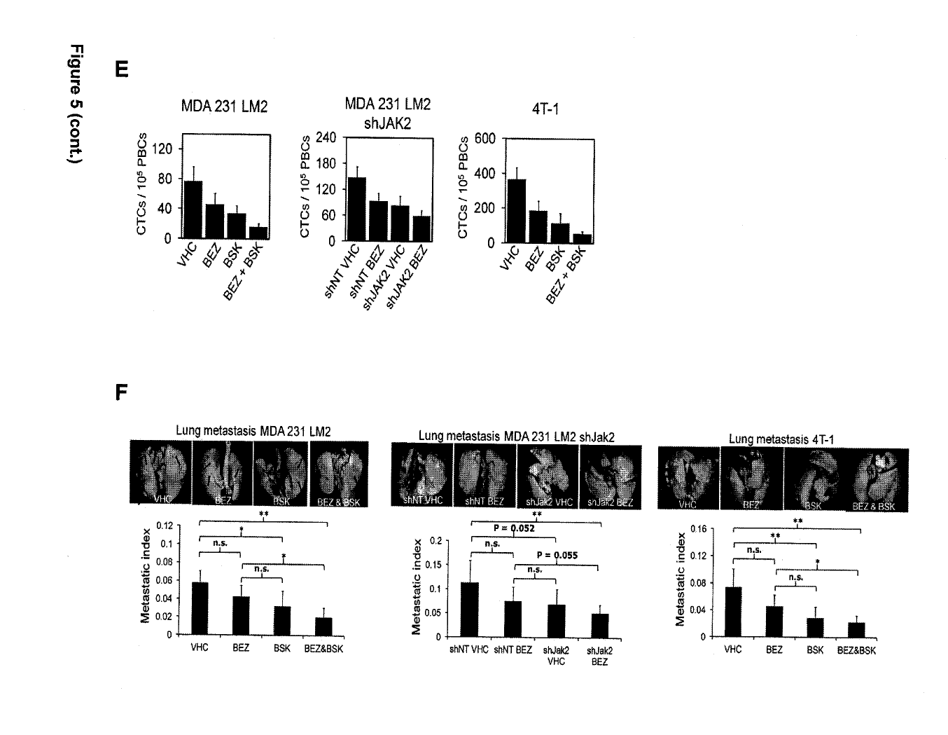

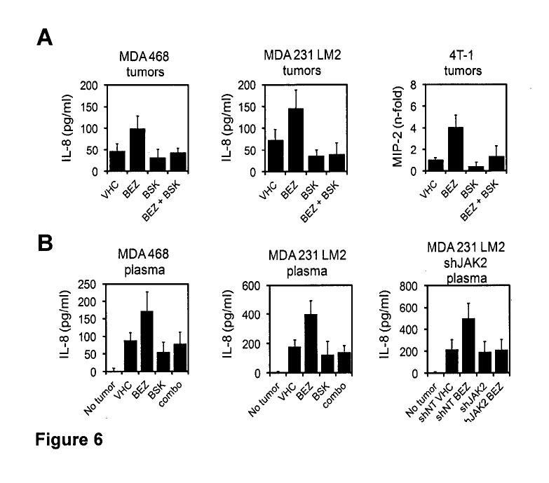

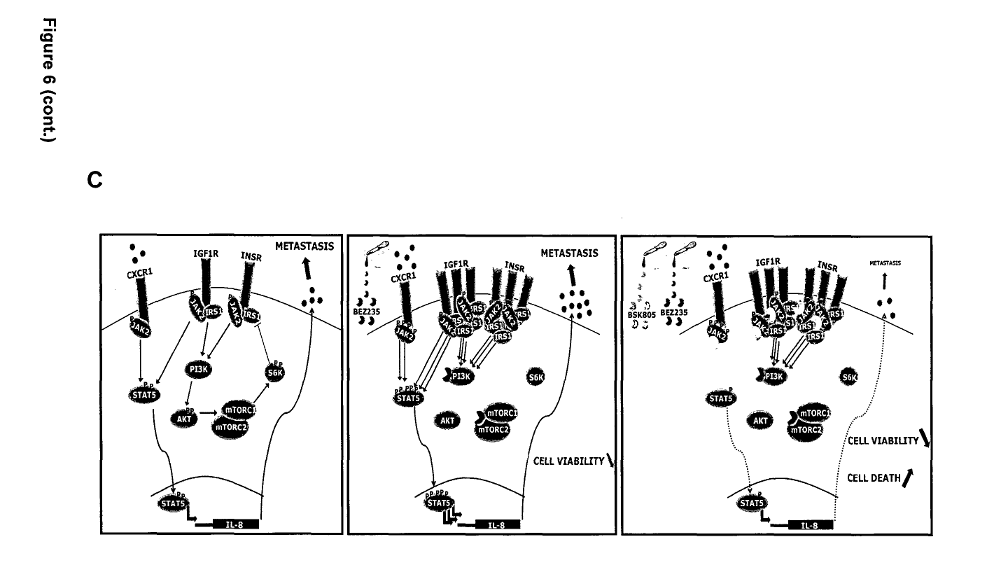

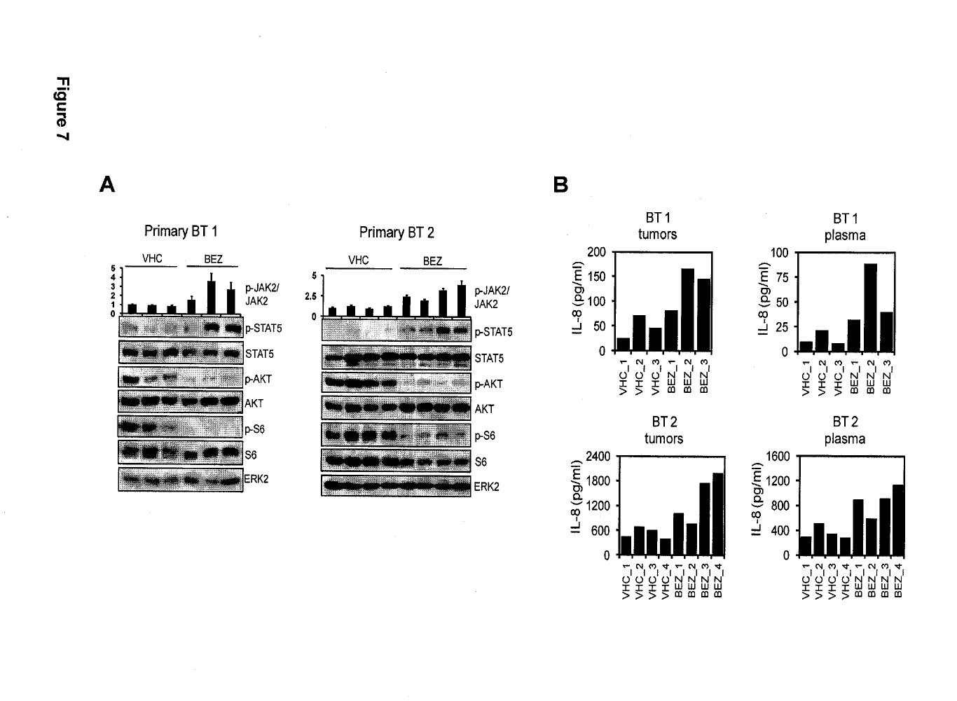

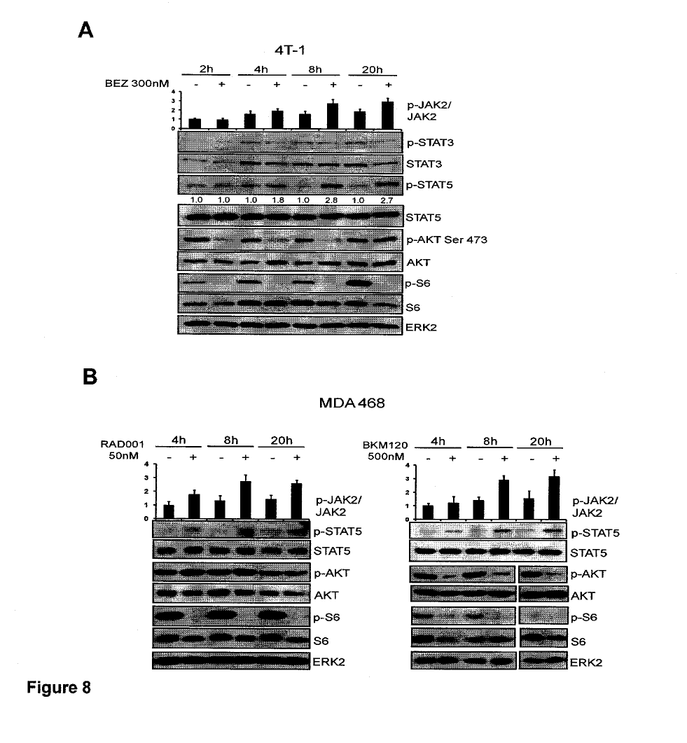

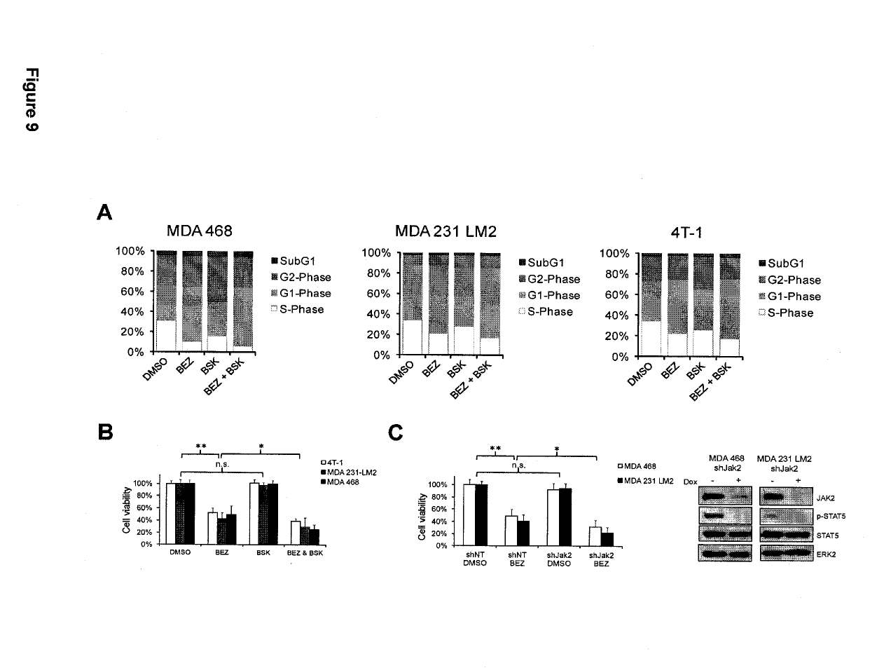

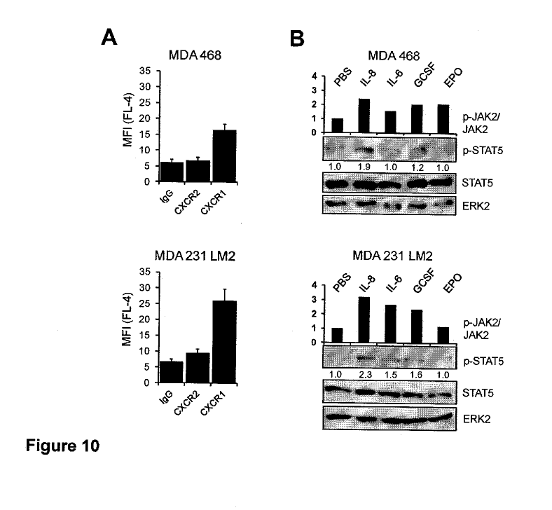

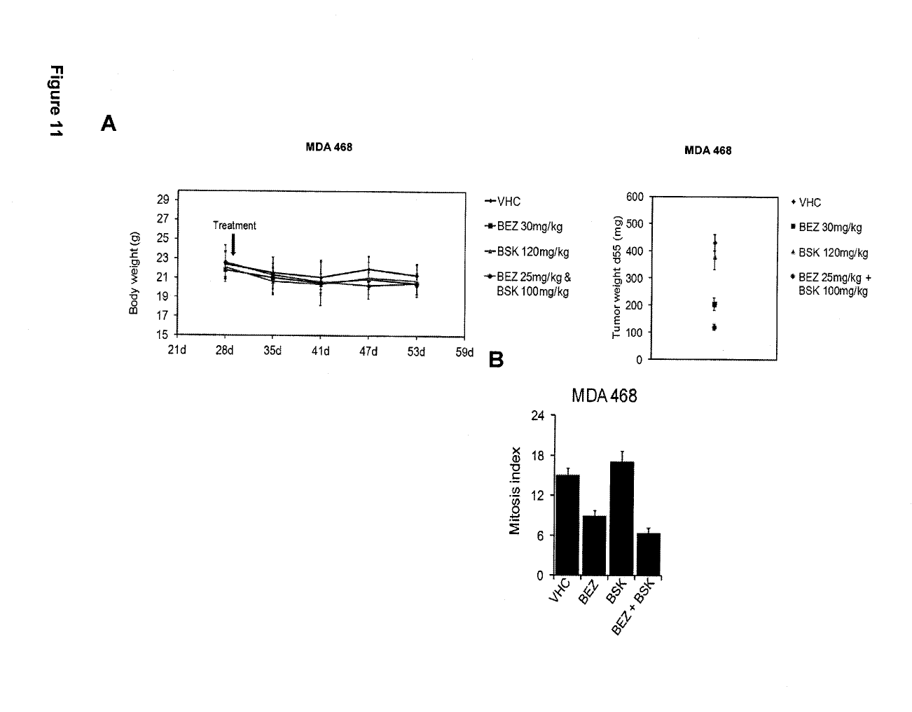

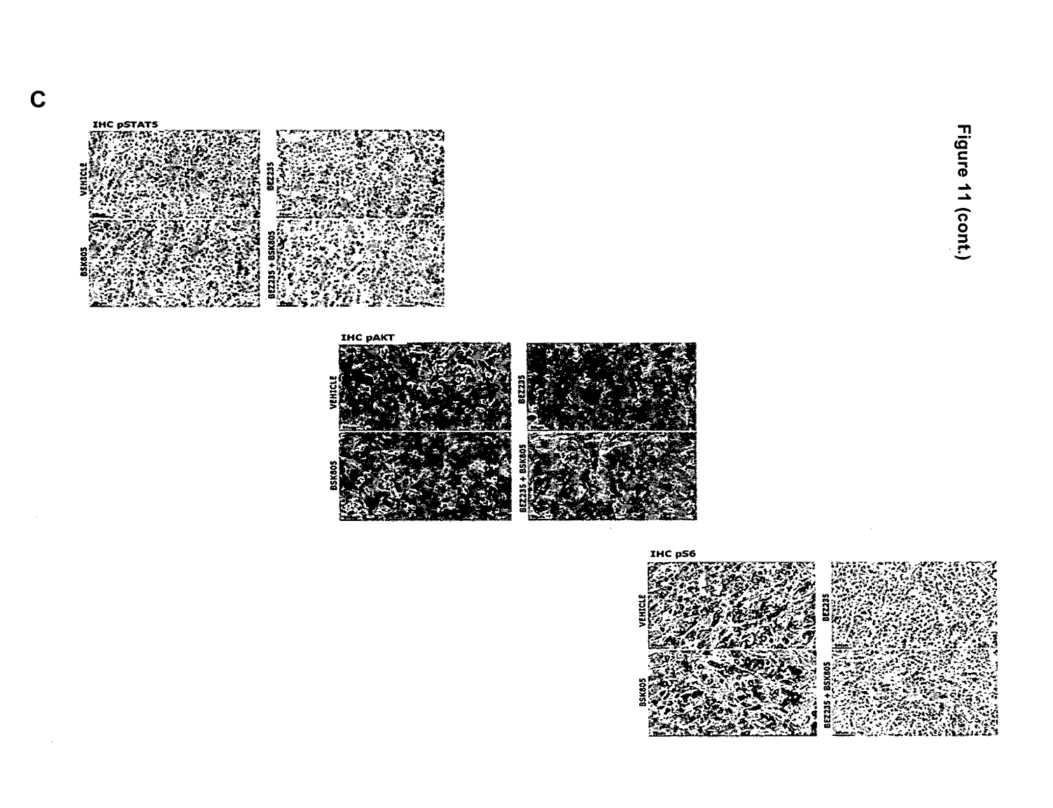

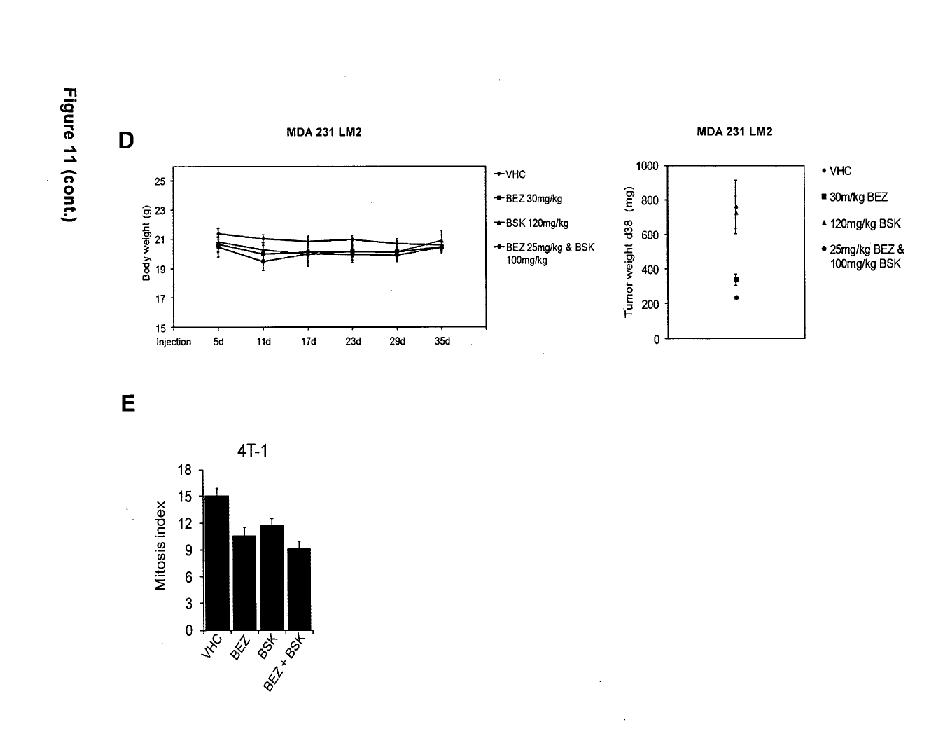

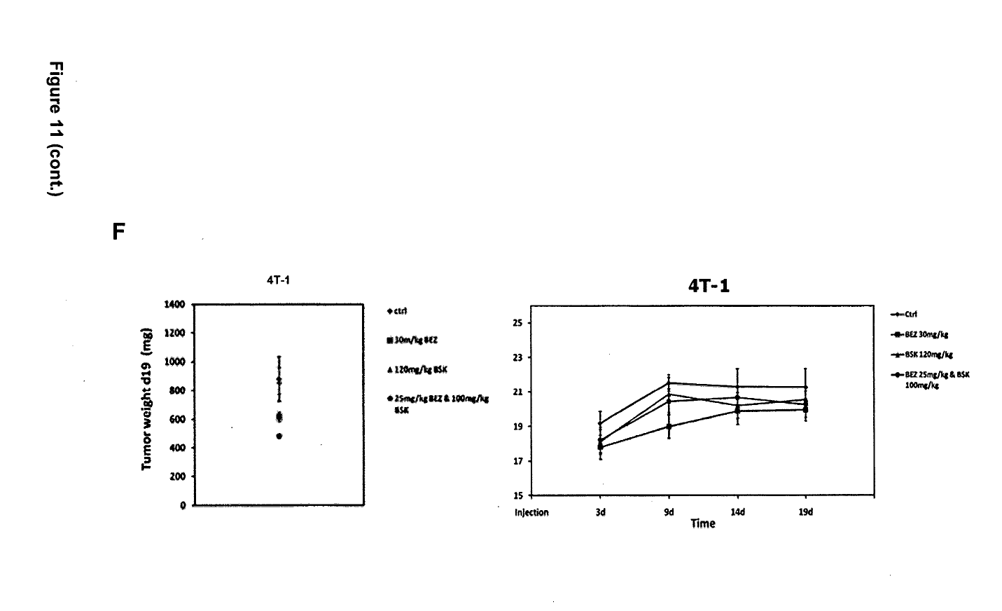

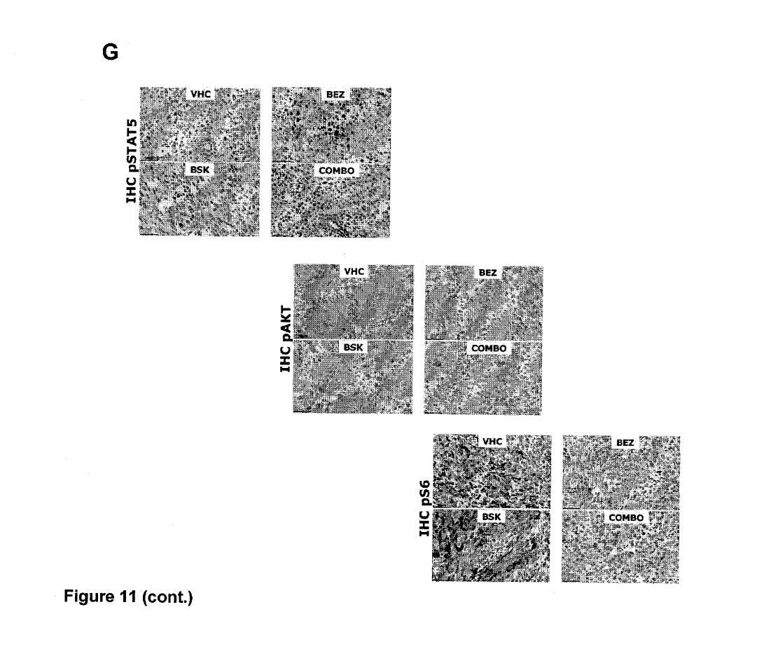

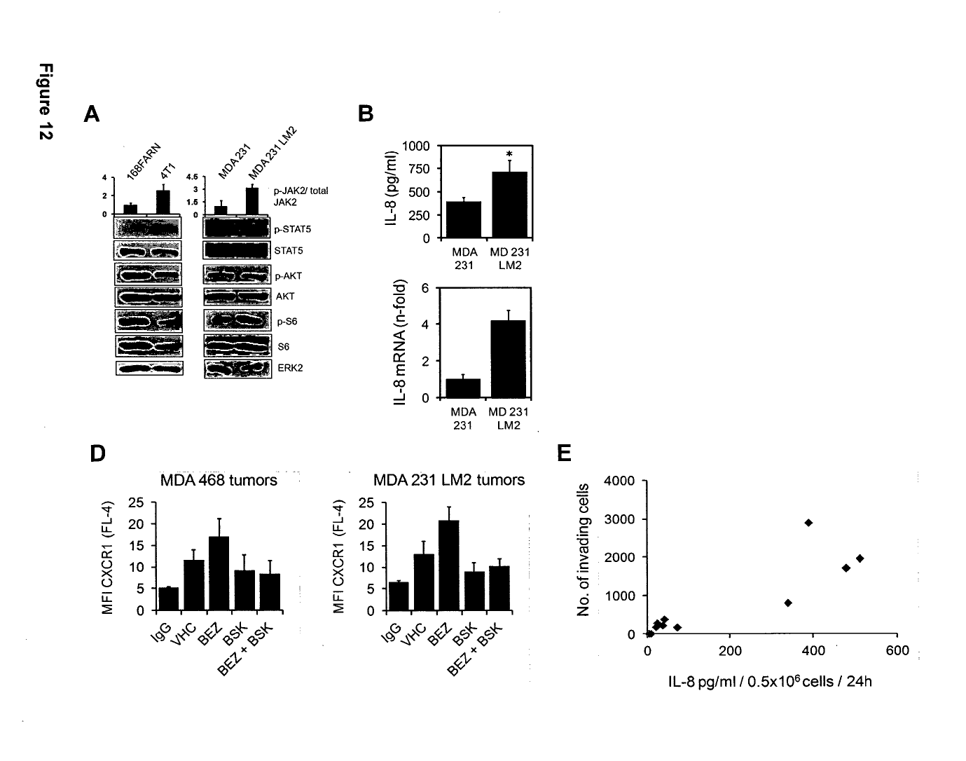

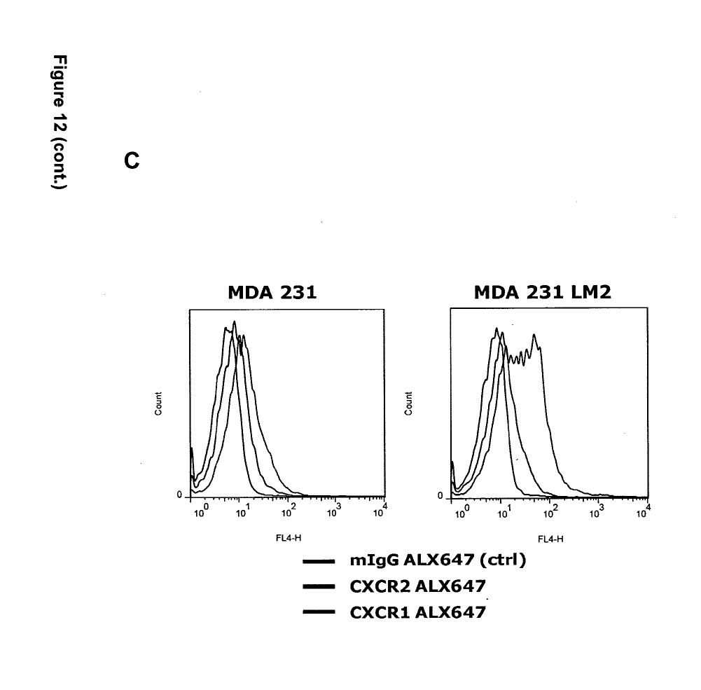

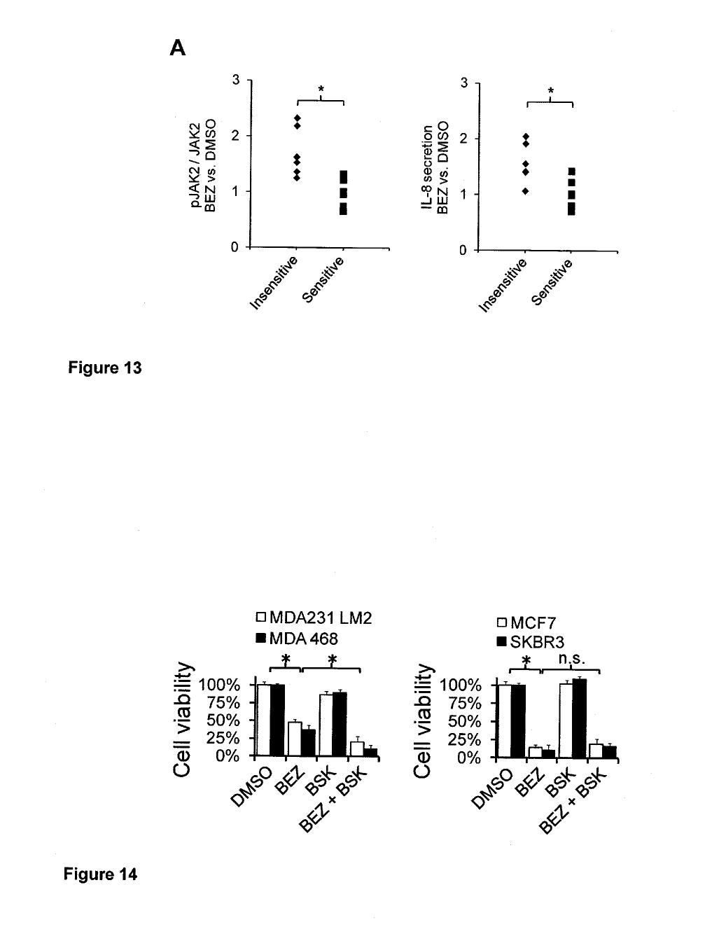

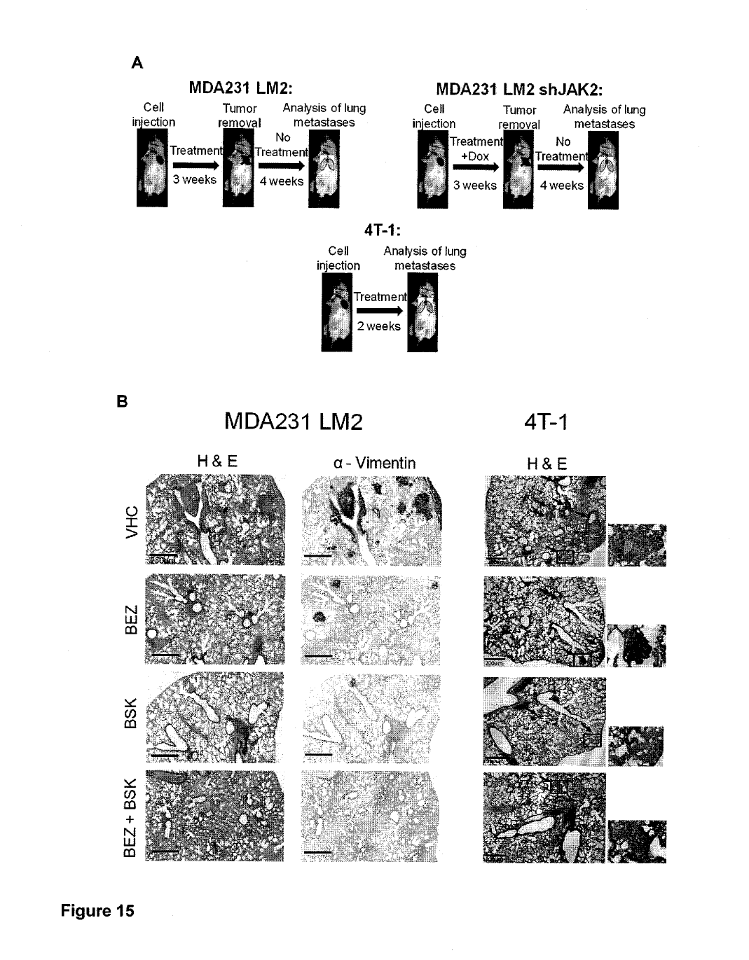

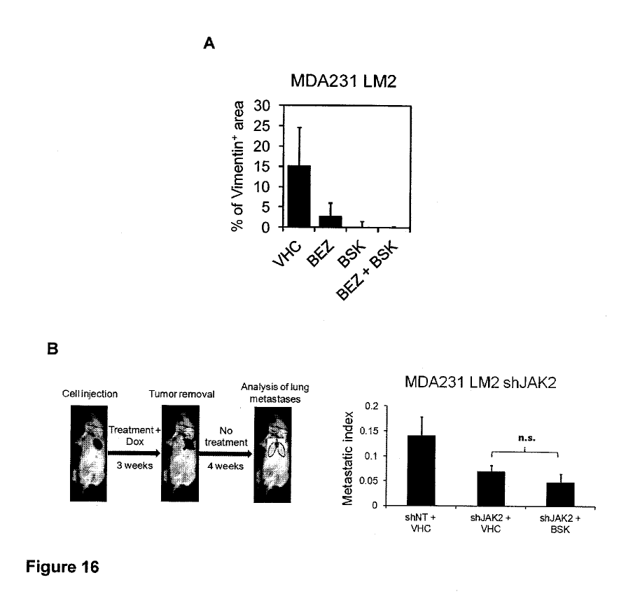

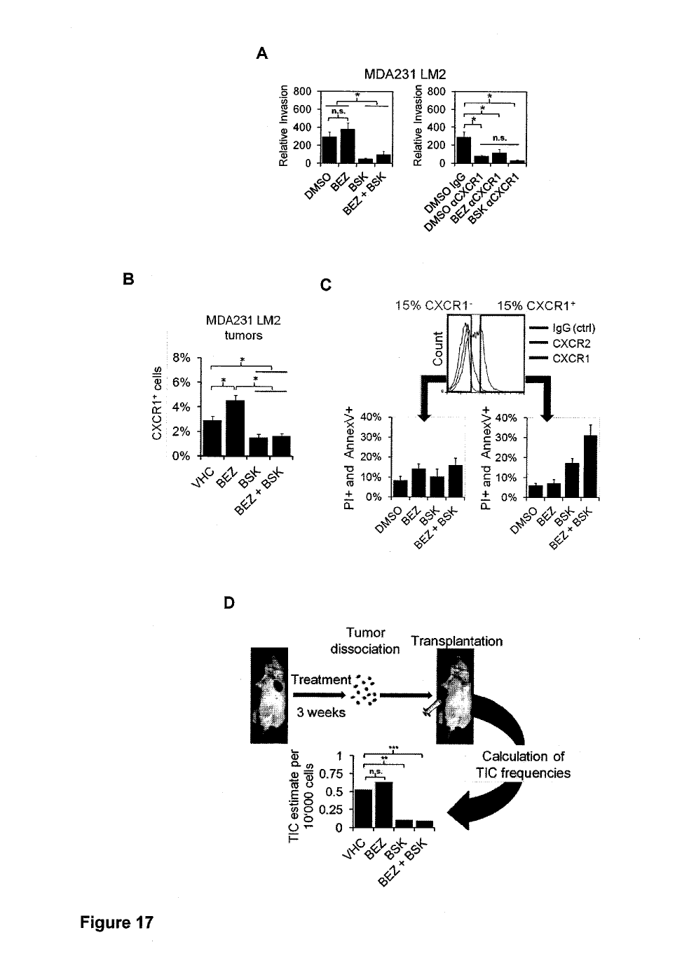

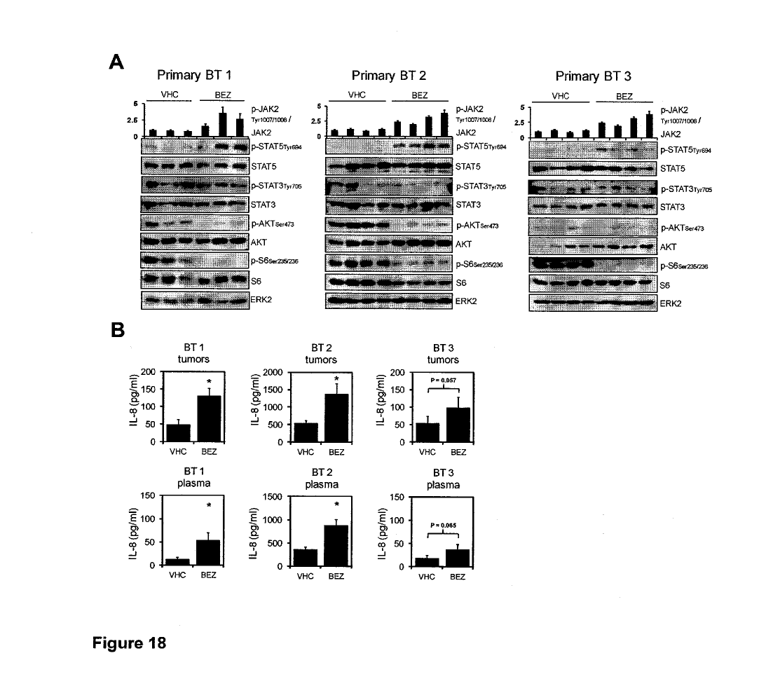

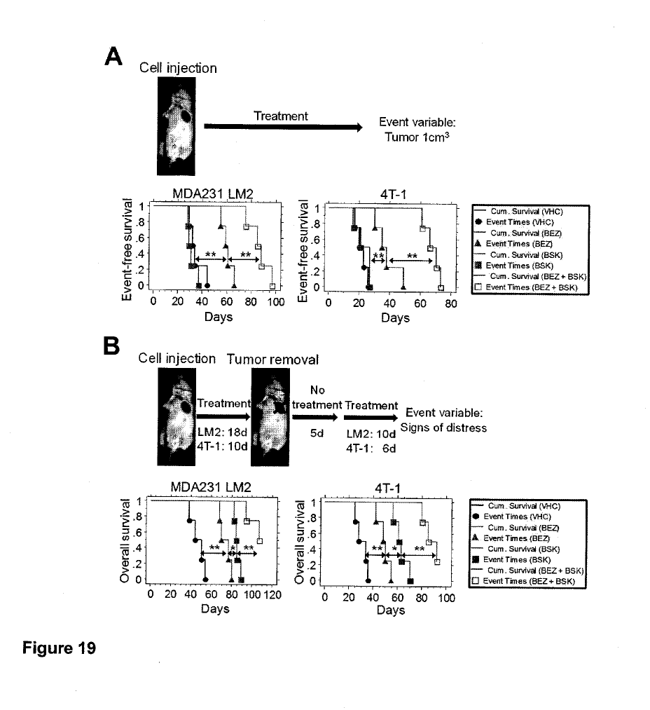

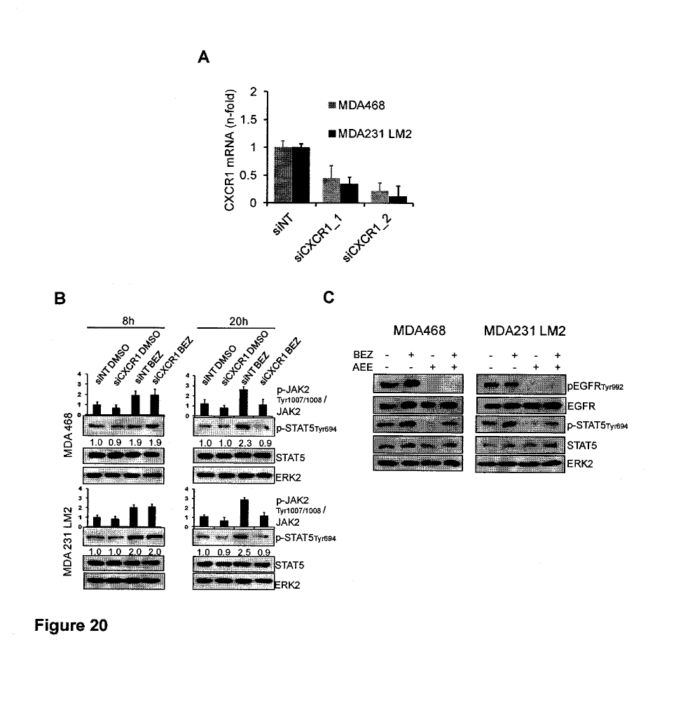

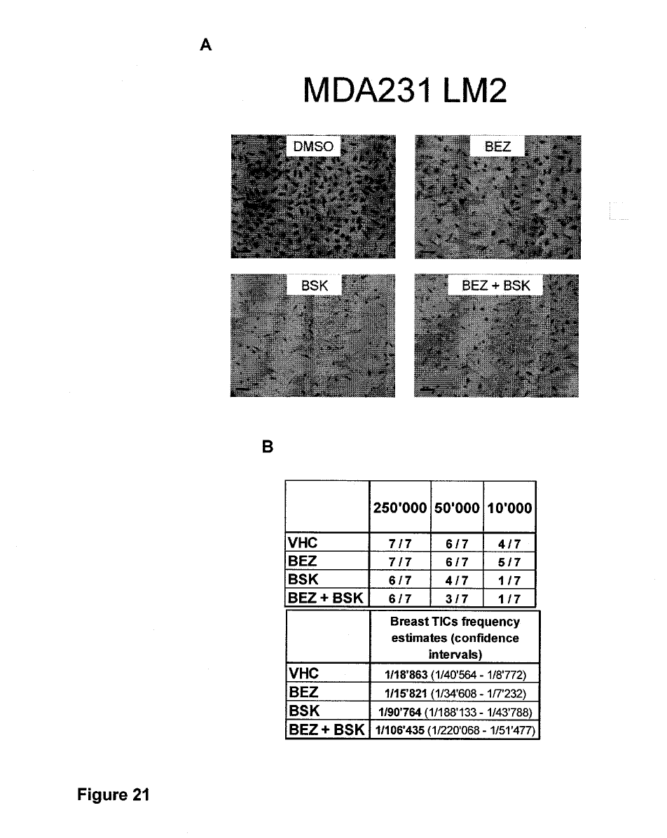

### EXAMPLES

Compounds and formulations NVP-BEZ235 (AN4) (PI3K/mTOR inhibitor), NVP-BSK805 (JAK2 inhibitor), NVP-BKM-120 (pan-PI3K inhibitor) and RAD001 (mTORC1 inhibitor) were all from Novartis, Basel, Switzerland. Compounds were prepared as 10 mmol/L stock solutions in DMSO and stored protected from light at −20° C. For dosing of mice, NVP-BSK805 was freshly formulated in NMP/PEG300/Solutol HS15 (5%/80%/15%), NVP-BEZ235 was freshly formulated in NMP/PEG300 (10%/90%) and both were applied at 10 mL/kg by oral gavage.

Cell lines, cell culture and in vitro experiments The lung metastatic subline of the parental MDA-MB-231, the MDA 231 LM2 (or 4175) was obtained from Joan Massagué (Memorial Sloan-Kettering Cancer Center, New York). MCF10A cells were cultured in DMEM/F12 (Invitrogen) supplemented with 5% Horse serum (Hyclone), 20 ng/ml of epidermal growth factor (EGF) (Peprotech), 0.5 μg/ml of Hydrocortisone (Sigma), 100 ng/ml of Cholera toxin (Sigma), 10 μg/ml of Insulin (Sigma), 100 IU/ml of penicillin and 100 μg/ml of streptomycin. SUM159PT cells were kindly provided by Charlotte Kuperwasser (Tufts University, Boston, Mass.) and were propagated in Ham's F12, 5% FCS, 1 μg/ml Insulin, 0.5 μg/ml Hydrocortisone. Balb/c lines 4T-1, 4T-1-GFP and 168FARN were provided by Nancy Hynes (FMI, Basel, Switzerland). All the other cell lines were from ATCC and culture conditions were according to the ATCC protocol. For treatment with inhibitors, cells were synchronized without serum o/n, then stimulated and treated as indicated. For experiments with doxycycline-inducible shRNAs, 500 ng/ml of doxycycline (Sigma) was added to the medium and experiments were started 48 h later to ensure efficient knockdown of the target. Cell viability in vitro was measured using the Cell Proliferation Reagent WST-1 (Roche). In brief, cells (2.5-4×10³) were plated in 96-well plates in quadruplicate in 200 μl normal growth medium and allowed to attach for 24 h prior to the addition of DMSO or inhibitors to the culture medium. After 72 h, 20 μl/well of the formazan dye was added. After incubation (4 h, 37° C., 5% CO₂ atmosphere), absorbance at 490 nm was recorded using an ELISA plate reader. Human cytokines Interleukin-6, Interleukin-8, GCSF and erythropoietin (EPO) were obtained from Peprotech and dissolved in PBS at 10 mg/ml/5000Units/ml for EPO. Cytokine stimulations were performed for 30 min with 10 ng/ml (10 Units/ml for EPO) with cells kept under low serum conditions. Antibody blocking experiments were performed with anti-CXCR1 (R&D, MAB330, 1 μg/ml), anti-CXCR2 (R&D, MAB331, 2.5 μg/ml) or a mouse IgG antibody (R&D, 1 μg/ml) for 45 min prior to lysis of the cells.

Immunoblotting and Immunoprecipitation Cells for Western Blotting and ELISA were lysed with RIPA buffer. Xenograft lysates were prepared by lysing kryo-homogenized tumor powder in RIPA buffer (50 mM Tris-HCl pH 8, 150 mM NaCl, 1% NP-40, 0.5% sodium deoxycholate, 0.1% SDS. RIPA was supplemented with 1× protease inhibitor cocktail (Complete Mini, Roche), 0.2 mmol/L sodium-vanadate, 20 mM sodium fluoride and 1 mmol/L phenylmethylsulfonyl fluoride. For IRS-1 immunoprecipitation, cell lysates containing 500-1000 μg of protein were incubated with 1 μg of antibody and 20-50 μl of protein A-Sepharose beads (Zymed Laboratories, Inc., South San Francisco, Calif.) overnight at 4° C. Immunoprecipitates or whole cell lysates (30-80 μg) were subjected to SDS-PAGE, transferred to PVDF membranes (Immobilon-P, Millipore) and blocked for 1 hr at room temperature with 5% milk in PBS-0.1% Tween 20. Membranes were then incubated overnight with antibodies as indicated and exposed to secondary HRP-coupled anti-mouse or -rabbit antibody at 1:5-10,000 for 1 h at room temperature. Proteins were visualized using an ECL kit (Amersham) or an enhanced chemiluminescence detection system (Pierce Biotechnology). In each of the studies presented, the results shown are typical of at least three independent experiments. The following antibodies were used: anti-JAK2 (Cell Signaling), anti-JAK1 (Cell Signaling), anti-pSTAT5 (Tyr694, Cell Signaling), anti-STAT5 (STAT5A&B, Cell Signaling), anti-STAT3 (Cell Signaling), anti-pSTAT3 (Tyr705, Cell Signaling), anti-AKT pan (Cell Signaling), anti-pAKT (Thr308 and Ser473, Cell Signaling), anti-ERK2 (Santa Cruz), anti-S6 (Cell Signaling), anti-pS6 (Ser235/236, Cell Signaling), anti-PARP (Cell Signaling), anti-MCL1 (Cell Signaling), anti-BIM (EL, L and S isoforms, Cell Signaling), anti-pIGF1R/plnsR (Invitrogen), anti-IGF1Rbeta (Cell Signaling), anti-InsRbeta (Santa Cruz), anti-IRS1 (Upstate), anti-pIRS1 (Tyr612, Calbiochem).

ELISA and Cytokine Arrays For assessing pJAK2 levels, an ELISA assay (Tyr1007/1008, Invitrogen) was applied because of cross-reactivity of all pJAK2 antibodies tested. Interleukin-8 levels in RIPA lysates, cell culture supernatants and mouse tail vein blood plasma were measured by ELISA, as well (Biolegend). Cytokine arrays on cell culture supernatants and mouse tumor lysates were performed according the manufacture's protocol (R & D systems, Human and Mouse cytokine array panel A).

RNA preparation and RQ-PCR Total RNA was extracted using the RNeasy Mini Kit and DNase elimination columns according to the manufacturer's protocol (Qiagen). 1 μg of total RNA were transcribed using the Thermo Script RT-PCR System from Invitrogen. PCR and fluorescence detection were performed using the StepOnePlus Sequence Detection System (Applied Biosystems, Rotkreuz, Switzerland) according to the manufacturer's protocol in a reaction volume of 20 μl containing 1× TaqMan® Universal PCR Master Mix (Applied Biosystems) and 25 ng cDNA. For quantification of IL-8, GAPDH and RPLPO mRNA, the 1× Taqman® Gene Expression Assays Hs00174103_m1, Hs02758991_g1 and Hs99999902_m1 (Applied Biosystems) were used. All measurements were performed in duplicates and the arithmetic mean of the Ct-values was used for calculations: target gene mean Ct-values were normalized to the respective housekeeping genes (GAPDH and RPSO), mean Ct-values (internal reference gene, Ct), and then to the experimental control. Obtained values were exponentiated 2(-ΔΔCt) to be expressed as n-fold changes in regulation compared to the experimental control (2(-ΔΔCt) method of relative quantification (Livak and Schmittgen, 2001).

Gene silencing procedures siRNAs were ordered as RP-HPLC purified duplexes from Sigma-Aldrich, the sequences were the following: siJAK1_{—}1 5′-GCACAGAAGACGGAGGAAAUGGUAU-3′ (SEQ ID NO:1), siJAK1_{—}2 5′-GCCUUAAGGAAUAUCUUCCAAAGAA-3′ (SEQ ID NO:2), si-IRS1: 5′-AACAAGACAGCUGGUACCAGG-3′ (SEQ ID NO:3), siNT (non-targeting control) 5′-AUUCUAUCACUAGCGUGACUU-3′ (SEQ ID NO:4). For JAK2, Validated Stealth RNAi™ siRNA were ordered from Sigma-Aldrich (VHS41246). Transfections of siRNAs were performed using according to the manufacture's guidelines (Dharma Fect 1, Dharmacon). For lentiviral production, 293T cells were plated at a density of 2.5×10⁶ cells per 10 cm culture dish. Cells were cotransfected by PEI method (PEI:DNA ratio=4:1) with either 15 μg of pLKO1-tet-on-JAK2 shRNA (#629, target sequence: TGGATAGTTACAACTCGGCTT (SEQ ID NO:5)) or pLK01-tet-on-non-silencing shRNA (Wiederschain et al., 2009) and 10 μg of 3^{rd} generation packaging plasmid mix. The culture medium was replaced with fresh medium after 16 hr. Supernatant was collected 48 and 72 hr after transfection. For determining the viral titers, 10⁵ MDA-MB-468 and MDA-MB-231-LM2 cells were seeded in a six-well plate and transduced with various dilutions of the vector in the presence of 8μ of Polybrene per milliliter (Sigma-Aldrich). The culture medium was replaced 72 hr later with fresh medium containing puromycin (Sigma-Aldrich) at a concentration of 1.5 μg/ml. MDA-MD-468 and MDA-MB-231-LM2 cells transduced with viral vector at a multiplicity of infection of 20 were used for experiments.

Flow cytometry Cells were detached using Trypsin-EDTA, resuspended in normal growth medium and counted. Tumors were mechanically and enzymatically dissociated (using collagenase II and HyQtase digestion). For Annexin V staining, 0.5×10⁶ cells were washed with cold PBS/5% BSA, resuspended in 70 μl binding buffer and labelled with phycoerythrin (PE)-labelled antibody against Annexin V according to the manufacturer's protocol (Becton Dickinson). For cell cycle analysis, 1×10⁶ cells were washed in PBS, fixed in 70% Ethanol for 60 min at 4° C., washed twice and resuspended in PI buffer (PBS supplemented with 50 μg/ml propidium iodide, 10 μg/ml RNAse A, 0.1% sodium citrate and 0.1% Triton X-100). For analysis of CXCR1 and CXCR2 cell surface expression, cells were incubated with 2.5 μg/10⁶ cells anti-CXCR1 (R&D, MAB330), anti-CXCR2 (R&D, MAB331) or with 1 μg/10⁶ cells mouse IgG antibody (R&D) for 20 min at 4° C., then with a secondary anti-mouse IgG-AlexaFluor647 (Biolegend) for 15 min at 4° C. in the dark prior to washing and analysis. At least 10⁴ cells per sample were analyzed with a FACScan flow cytometer (Becton Dickinson, Basel, Switzerland).

Animal experiments. SCID/beige, SCID/NOD and Balb/c mice (Jackson Labs) were maintained under specific pathogen-free conditions and were used in compliance with protocols approved by the Institutional Animal Care and Use Committees of the FMI, which conform to institutional and national regulatory standards on experimental animal usage. For orthotopical engraftment of breast cancer cell lines, 1×10⁶ MDA-MB-468, 1×10⁶ MDA-MB-231-LM2 and 0.5×10⁶ 41-1 or 4T-1-GFP cells were suspended in a 100-μl mixture of Basement Membrane Matrix Phenol Red-free (BD Biosciences) and PBS 1:1 and injected into the mammary gland 4 or between mammary glands 2 and 3. Primary patient breast tumors were cut into 1 mm×1 mm pieces and transplanted into mammary gland 4. Tumor-bearing mice were randomized based on tumor volume prior to the initiation of treatment, which was initiated when average tumor volume was at least 100 mm³. BEZ-235 and BKS-805 were given orally (formulations see above) on each of 6 consecutive days followed by one day of drug holiday. Expression of shRNAs was induced by adding doxycycline in the drinking water (2 g/l of in a 5% sucrose solution), which was refreshed every 48 h. Tumors were measured every 3 to 4 days with vernier calipers, and tumor volumes were calculated by the formula 0.5×(larger diameter)×(smaller diameter)². End point tumor sizes were analyzed for synergism using the formula AB/C<A/C×B/C, where C=tumor volume VHC, A=tumor volume compound 1, B=tumor volume compound 2, AB=tumor volume combination (Clarke, 1997).

Immunohistochemistry. Tumors were fixed in 10% NBF (Neutral buffered formalin) for 24 h at 4° C., washed with 70% EtOH, embedded in paraffin and stained with H&E, anti-Ki67 (Thermo Scientific), anti-pSTAT5 (Tyr694, cell signaling), anti-pAKT (Ser473, cell signaling), anti-pS6 (Ser235/236, cell signaling), anti-PARP (cell signaling) and anti-mouse F4/80 (AbD Serotec) antibodies. Mouse lungs were fixed in Bouin's fixative and visible metastatic lung nodules were counted using a binocular.

Statistical analysis. Each value reported represents the mean±s.d. or s.e. of at least three independent experiments. Data were tested for normal distribution and Student's t-test or nonparametric Mann-Whitney U-tests were applied using the program JMP4 (SAS, Cary, N.C., USA). P-values<0.05 were considered to be statistically significant.

The present inventors applied single doses of COMPOUND A, a dual PI3K and mTOR inhibitor, and analyzed target inhibition and potential signaling pathway crosstalks after 2, 4, 8 and 20 h hours of treatment. They found that COMPOUND A reduced pAKT and completely blocked pS6 levels up to 20 hours after treatment in the PTEN-deficient MDA 468 and the RAS-mutated MDA 231 LM2 breast cancer lines, as well as in the mouse breast cancer line 4T-1. The present inventors further used in vivo models to confirm these results. Surprisingly, they detected a considerable upregulation of pJAK2 and pSTAT5 after 4 hours-8 hours of BEZ treatment in vitro and after 8 hours of treatment in vivo. Levels of pSTAT3 remained however largely unaffected by BEZ treatment. In order to elucidate which arm of the dual inhibitor COMPOUND A could be responsible for the observed crosstalk to JAK2, the present inventors used a PI3K-specific inhibitor (BKM120) and an mTOR inhibitor (RAD001). They found that both single inhibition of PI3K and mTOR upregulated pJAK2 and pSTAT5, however at different time points. While RAD001 readily activated JAK2 starting at 4 hours of treatment, they observed a later response with BKM120 treatment starting at 8 hours after adding the compound. Given the fact that both JAK2 and JAK1 are capable of signaling to STAT5 and STAT3 depending on the cell type and the receptor they are associated with (Desrivières et al., 2006; Bezbradica et al., 2009), the present inventors performed siRNA depletion of both JAKs and found that only JAK2 is responsible for activation of STAT5 while JAK1 is upstream of STAT3 in the experimental models used. Next, they investigated whether JAK2 activation is necessary for upregulation of pSTAT5 by BEZ treatment and if a highly specific JAK2 inhibitor, COMPOUND D (Radimerski et al, 2010), would be sufficient to block this crosstalk. The results show that both siRNA depletion of JAK2 and inhibition of its activity counteracted upregulation of pSTAT5 bp BEZ. Hence, the inventors found a JAK2/STAT5-evoked positive feedback loop that causes resistance to dual PI3K/mTOR inhibition. Mechanistically, PI3K/mTOR inhibition increased IRS1-dependent activation of JAK2/STAT5 and secretion of IL-8 in several cell lines and primary triple-negative breast cancer. Genetic or pharmacological inhibition of JAK2 abrogated this feedback loop. They further showed that combined PI3K/mTOR and JAK2 inhibition synergistically reduced cancer cell number in vitro, as well as tumor growth, the number of circulating tumor cells and metastasis in vivo. The inventors' study thus revealed a new link between growth factor signaling, JAK/STAT activation and cytokine secretion. Their results provide a rationale for combined targeting of the PI3K/mTOR and JAK2/STAT5 pathways in proliferative diseases.

TABLE 1
BEZ increased phosphorylation of JAK2/STAT5 and IL-8 secretion in a
panel of breast cancer cell lines. Shown are the levels of JAK2/STAT5
phosphorylation and IL-8 secretion upon treatment of triple-negative
(bold) and luminal (grey) breast cancer cell lines with 300 nM BEZ for 8 h
or 20 h, respectively. pSTAT5/STAT5 levels were assessed by
immunoblotting and quantified by densitometry. pJAK2/JAK2 and
IL-8 levels were measured by ELISA. Values from BEZ-treated relative
to DMSO cells are given. Data are presented as
mean ± SD (n = 3).

| | pJAK2/JAK2 | pSTAT5/STAT5 | IL8 |
|---|---|---|---|
| | BEZ vs. DMSO | BEZ vs. DMSO | BEZ vs. DMSO |
| MDA 468 | 2.32 | 2.11 | 1.91 |
| MDA 231 | 1.25 | 1.58 | 1.41 |
| MDA 231 | 2.18 | 2.32 | 2.04 |
| LM2 | | | |
| HCC1954 | 1.32 | 1.05 | 1.22 |
| Hs578t | 1.18 | 0.96 | 1.08 |
| BT549 | 1.21 | 1.61 | 1.42 |
| SUM159 | 0.92 | 1.09 | 1.24 |
| HCC1937 | 1.62 | 1.66 | 1.06 |
| MDA 436 | 1.53 | 1.77 | 1.41 |
| MCF10A | 2.11 | 3.21 | 1.23 |
| 4T-1 | 2.02 | 2.65 | n.d. |
| 168FARN | 1.74 | 1.9 | n.d. |
| ZR75-1 | 1.36 | 1.62 | 1.55 |
| T47D | 1.05 | 0.62 | 0.71 |
| MCF7 | 0.74 | 0.92 | 0.81 |
| BT474 | 0.95 | 1.02 | 0.99 |
| MDA415 | 0.88 | n.d. | 0.91 |
| SKBR3 | 0.65 | 0.82 | 1.01 |

1. A combination for use as a medicament, which combination comprises (a) phosphoinositide 3-kinase (PI3K) inhibitor compound and (b) a compound which modulates the Janus Kinase 2 (JAK2)—Signal Transducer and Activator of Transcription 5 (STAT5) pathway, wherein the active ingredients are present in each case in free form or in the form of a pharmaceutically acceptable salt or any hydrate thereof, and, optionally at least one pharmaceutically acceptable carrier; for simultaneous, separate or sequential use.

2. A combination according to claim 1 wherein the phosphoinositide 3-kinase inhibitor compound is selected from the group consisting of COMPOUND A, COMPOUND B, COMPOUND C, rapamycin, temsirolimus, everolimus, temsirolimus, ridaforolimus, MK-8669, sirolimus, zotarolimus and biolimus.

3. A combination according to claim 1 wherein the compound which modulates the JAK2-STAT5 pathway is selected from the group consisting of Lestaurtinib, Ruxolitinib, SB1518, CYT387, LY3009104, INC424, LY2784544, BMS-911543, NS-018, TG101348, COMPOUND D, COMPOUND E, COMPOUND F, COMPOUND G, COMPOUND H and COMPOUND I.

4. A combination according to claim 1 wherein the phosphoinositide 3-kinase inhibitor compound and/or the compound which modulates the JAK2-STAT5 pathway is a siRNA.

5. A combination according to claim 1 wherein the compound which modulates the JAK2-STAT5 pathway inhibits the secretion of interleukin 8 (IL8).

6. A combination according to claim 1 wherein the phosphoinositide 3-kinase inhibitor is COMPOUND A.

7. A combination according to claim 1 wherein the phosphoinositide 3-kinase inhibitor is COMPOUND C.

8. A combination according to claim 1 wherein the phosphoinositide 3-kinase inhibitor is everolimus.

9. A combination according to claim 1 for use in the treatment of a proliferative disease.

10. A combination according to claim 1 for use in the treatment of a solid tumor.

11. A combination according to claim 1 for use in the treatment of a breast cancer.

12. A combination according to claim 1 for use in the treatment of a metastatic breast cancer.

13. A combination according to claim 1 for use in the treatment of a triple-negative breast cancer,

14. A combination according to claim 1, wherein said preparation comprises (a) one or more unit dosage forms of phosphoinositide-3 kinase inhibitor (PI3K) and (b) one or more unit dosage forms of a compound which modulates the JAK2-STAT5 pathway.


SEQUENCE LISTING (5 sequences)

SEQ ID NO: 1 - RNA, 25 nt, Homo sapiens
  source 1..25: /mol_type=“RNA” /organism=“Homo sapiens”
gcacagaagacggaggaaaugguau

SEQ ID NO: 2 - RNA, 25 nt, Homo sapiens
  source 1..25: /mol_type=“RNA” /organism=“Homo sapiens”
gccuuaaggaauaucuuccaaagaa

SEQ ID NO: 3 - RNA, 21 nt, Homo sapiens
  source 1..21: /mol_type=“RNA” /organism=“Homo sapiens”
aacaagacagcugguaccagg

SEQ ID NO: 4 - RNA, 21 nt, Homo sapiens
  source 1..21: /mol_type=“RNA” /organism=“Homo sapiens”
auucuaucacuagcgugacuu

SEQ ID NO: 5 - DNA, 21 bp, Homo sapiens
  source 1..21: /mol_type=“DNA” /organism=“Homo sapiens”
tggatagttacaactcggctt